(19) United States
(12) Patent Application Publication
(10) Pub. No.: US 2024/0120027 A1
(43) Pub. Date: Apr. 11, 2024

(54) MACHINE-LEARNING MODEL FOR REFINING STRUCTURAL VARIANT CALLS

(71) Applicant: Illumina, Inc., San Diego, CA (US)

(72) Inventors: Sujai Chari, Burlingame, CA (US); Gavin Derek Parnaby, Laguna Niguel, CA (US); Naoki Nariai, San Diego, CA (US)

(21) Appl. No.: 18/476,232

(22) Filed: Sep. 27, 2023

Related U.S. Application Data
(60) Provisional application No. 63/377,846, filed on Sep. 30, 2022.

(51) Int. Cl. G16B 20/20; G06N 20/20; G16B 20/10; G16B 40/20

(52) U.S. Cl. CPC G16B 20/20; G06N 20/20; G16B 20/10; G16B 40/20

(57) ABSTRACT

This disclosure describes methods, non-transitory computer readable media, and systems that can utilize a machine-learning model to refine structural variant calls of a call generation model. For example, the disclosed systems can train and utilize a structural variant refinement machine-learning model to reduce false positives and/or false negatives. Indeed, the disclosed systems can improve or refine structural variant calls (e.g., between 50-200 base pairs in length) determined by a call generation model by training and utilizing the structural variant refinement machine-learning model. As disclosed, the systems can determine sequencing metrics and can customize training data for a structural variant refinement machine-learning model to generate modified structural variant calls.

### CROSS-REFERENCE TO RELATED APPLICATIONS

The present application claims the benefit of, and priority to, U.S. Provisional Application No. 63/377,846, entitled “MACHINE-LEARNING MODEL FOR REFINING STRUCTURAL VARIANT CALLS,” filed on Sep. 30, 2022. The aforementioned application is hereby incorporated by reference in its entirety.

### BACKGROUND

In recent years, biotechnology firms and research institutions have improved hardware and software for sequencing nucleotides and determining nucleotide base calls for genomic samples. For instance, some existing sequencing machines and sequencing-data-analysis software (together “existing sequencing systems”) predict individual nucleotide bases within sequences by using conventional Sanger sequencing or sequencing-by-synthesis (SBS) methods. When using SBS, existing sequencing systems can monitor many thousands of oligonucleotides being synthesized in parallel from templates to predict nucleotide base calls for growing nucleotide reads. In many existing sequencing systems, a camera captures images of irradiated fluorescent tags incorporated into oligonucleotides. After capturing such images, some existing sequencing systems determine nucleotide base calls for nucleotide reads corresponding to the oligonucleotides and send base-call data to a computing device with sequencing-data-analysis software, which aligns nucleotide reads with a reference genome. Based on differences between the aligned nucleotide reads and the reference genome, existing systems can further utilize a variant caller to identify variants of a genomic sample, such as single nucleotide polymorphisms (SNPs), and/or structural variants.

Despite these recent advances in sequencing and variant calling, existing sequencing systems often include variant callers that inaccurately determine structural variant calls, especially for structural variants within a threshold range of base-pair length (e.g., from 50-200 base pairs in length). For example, many existing systems generate structural variant calls that include excessive numbers of false positive calls and/or false negative calls for structural variants within a threshold range of base-pair length. Contributing to this inaccuracy, some existing sequencing systems are overly reliant on unreliable truth set data. For instance, some existing systems perform variant calling and/or variant call filtering based on data that contain certain inconsistencies and errors, such as inconsistent or error-prone read data or inconsistent or error-prone reference data from sequencing processes and/or variant calls from variant calling models. Indeed, standard or replacement truth set data in the industry (e.g., precisionFDA truth set data or long-read data) contains errors or read-coverage holes (however small in number) that can propagate through and affect structural variant calling for existing systems trained on these data. Consequently, relying on such truth set data too heavily results in many existing systems generating structural variant calls that include excessive numbers of false positive calls and/or false negative calls that could otherwise be reduced with a more accurate system. As described below, truth set data has proven particularly problematic for existing sequencing systems determining relatively smaller size structural variant calls within a threshold range of base-pair lengths.

To compound such structural-variant-calling inaccuracy, some existing sequencing systems utilize models that require training on millions or billions of base-call data that is either unavailable or incomplete. More specifically, some sequencing systems utilize deep learning models that require an excessive amount of training data to achieve acceptable measures of accuracy. However, training data for structural variants is relatively limited across the industry, and training models using incomplete or insubstantial data results in inaccurate and unreliable structural variant call predictions. Thus, existing systems that rely on deep learning models often produce inaccurate structural variant calls that can be especially pronounced for relatively smaller size structural variants within a threshold range of base-pair length.

In addition to inaccurately determining structural variant calls, some existing sequencing systems also inefficiently expend computing resources with overly complex models. Specifically, the structural variant callers of some existing sequencing systems are computationally expensive and slow. Indeed, some existing sequencing systems utilize structural variant callers with deep learning architectures that require extensive computational resources (e.g., computing time, processing power, and memory) to train and apply. For example, some existing sequencing systems utilize deep learning architectures that, even after training, consume many hours across multiple computing devices to generate structural variant calls for a single sample sequence.

As an added drawback of existing sequencing systems with complex deep learning networks, many such systems utilize model architectures that render sequence data uninterpretable. More specifically, some existing deep neural networks for variant calling transform and manipulate the sequence data many times over, changing from one uninterpretable latent vector to another such latent vector across the various layers and neurons, as the basis for generating a structural variant call. In many cases, the internal data of these deep neural networks is uninterpretable and impossible to utilize in any way outside of the neural network architecture itself.

### SUMMARY

This disclosure describes embodiments of methods, non-transitory computer readable media, and systems that can utilize a machine-learning model to modify or confirm structural variant calls of a call generation model. For example, the disclosed systems can train or utilize a structural variant refinement machine-learning model to reduce false positive calls (e.g., structural variant calls where no structural variant exists) and/or false negative calls (e.g., no structural variant call where a structural variant exists). Indeed, the disclosed systems can determine sequencing metrics corresponding to an initial structural variant call and utilize the structural variant refinement machine-learning model to determine, based on the sequencing metrics, a false positive likelihood that the initial structural variant call is a false positive. Based on the false positive likelihood from the structural variant refinement machine-learning model, the disclosed systems can correct or confirm structural variant calls (e.g., between 50-200 base pairs in length) initially determined by a call generation model. As disclosed, the systems can also customize or correct training data for structural variants to train a structural variant refinement machine-learning model to generate modified structural variant calls.

### BRIEF DESCRIPTION OF THE DRAWINGS

The detailed description refers to the drawings briefly described below.

FIG. 1 illustrates a block diagram of a sequencing system including a call refinement system in accordance with one or more embodiments.

FIG. 2 illustrates an overview of the call refinement system generating a modified structural variant call using a structural variant refinement machine-learning model in accordance with one or more embodiments.

FIG. 3 illustrates an example diagram of the call refinement system determining and utilizing sequencing metrics for use by a structural variant refinement machine-learning model to generate a false positive likelihood in accordance with one or more embodiments.

FIG. 4 illustrates the call refinement system generating false positive likelihoods and refining a structural variant call utilizing a structural variant refinement machine-learning model in accordance with one or more embodiments.

FIG. 5 illustrates an example table of in the call refinement system improving determinations of false positive structural variant calls utilizing a structural variant refinement machine-learning model in accordance with one or more embodiments.

FIG. 6 illustrates an example diagram for training a structural variant refinement machine-learning model in accordance with one or more embodiments.

FIG. 7 illustrates an example chart depicting correction of truth data for training a structural variant refinement machine-learning model in accordance with one or more embodiments.

FIG. 8 illustrates an example graph of results from cross validation training across different training datasets in accordance with one or more embodiments.

FIG. 9 illustrates an example graph comparing performance of different architectures of the structural variant refinement machine-learning model and performance of a call generation model in accordance with one or more embodiments

FIG. 10 illustrates an example graph of importance measures for different sequencing metrics for a structural variant refinement machine-learning model in accordance with one or more embodiments.

FIG. 11 illustrates a flowchart of a series of acts for generating a modified structural variant call utilizing a structural variant refinement machine-learning model in accordance with one or more embodiments.

FIG. 12 illustrates a block diagram of an example computing device for implementing one or more embodiments of the present disclosure.

### DETAILED DESCRIPTION

This disclosure describes embodiments of a call refinement system that generates and modifies structural variant (“SV”) calls for a genomic sample utilizing a structural variant refinement machine-learning model. In particular, the call refinement system can utilize a structural variant refinement machine-learning model to update, recalibrate, or modify an initial structural variant call (e.g., having a length between 50 and 200 base pairs) generated by a call generation model. In some cases, the call refinement system determines or identifies specific sequencing metrics (e.g., from read data, reference data, and/or base-call-quality data) to input into the structural variant refinement machine-learning model for generating a structural variant call. For instance, the call refinement system determines various types of sequencing metrics, such as read-based sequencing metrics, reference-based sequencing metrics, and variant region quality sequencing metrics. The call refinement system can further train or apply the structural variant refinement machine-learning model according to the sequencing metrics to generate modified (or refined or recalibrated) structural variant calls.

As just mentioned, in certain implementations, the call refinement system improves structural variant calls, such as structural variant calls having a number of base pairs less than a threshold length (e.g., 200 base pairs or some other threshold) or having a number of base pairs within a length window (e.g., 50-200 base pairs or some other window). To facilitate generating improved structural variant calls, in some embodiments, the call refinement system utilizes a structural variant refinement machine-learning model that is specialized to generate or predict structural variant calls at genomic coordinates or regions of a genomic sequence (e.g., genomic sample). Based on its training, the structural variant refinement machine-learning model is tailored to filter or refine initial structural variant calls (as generated by a call generation model) as a post processing analysis. In filtering or refining structural variant calls, the call refinement system can improve call accuracy and quality by reducing numbers of false positives and false negatives resulting from the structural variant calls of the call generation model.

As mentioned above, in some embodiments, the call refinement system determines confirmed or modified structural variant calls based on sequencing metrics analyzed by a machine-learning model. In particular, the call refinement system can extract, identify, or determine sequencing metrics to input into a structural variant refinement machine-learning model, whereupon the model generates a predicted structural variant call. For instance, the call refinement system can extract or determine sequencing metrics belonging to one or more categories, including: 1) read-based sequencing metrics, 2) reference-based sequencing metrics, and 3) variant region quality sequencing metrics. To determine or extract such sequencing metrics, the call refinement system can select metrics associated with a reference genome, metrics associated with read data obtained via SBS sequencing, and/or metrics associated with an initial variant call obtained via a call generation model (e.g., a DRAGEN SV caller). Additional detail regarding the makeup and determination of sequencing metrics is provided below with reference to the figures.

As further mentioned, in certain implementations, the call refinement system generates one or more structural variant calls for modifying or improving structural variant calls or variant call data fields of a variant call format (“VCF”) file. More specifically, the call refinement system utilizes a structural variant refinement machine-learning model to generate, from the sequencing metrics and an initial structural variant call, a false positive likelihood indicating a likelihood that the initial structural variant call (as determined via a call generation model) is a false positive. From the false positive likelihood, the call refinement system can further determine a modified structural variant call by, for instance, updating or modifying the initial structural variant call to indicate whether the genomic coordinates associated with the call reflect a structural variant (according to the false positive likelihood).

In one or more embodiments, the call refinement system further determines or generates training data for training a structural variant refinement machine-learning model. In particular, the call refinement system can modify a truth dataset to correct errors or inconsistencies and can use the corrected truth dataset as training data for the structural variant refinement machine-learning model. In some cases, the call refinement system detects or identifies errors in a truth dataset and automatically corrects the errors, such as missed (or incorrectly labeled) structural variant calls from a Circular Consensus Sequencing (CCS) Read-Based SV caller. Using the corrected data for more accurate training, the call refinement system can train the structural variant refinement machine-learning model for more precise structural variant calling, reducing false positives and false negatives.

As suggested above, the call refinement system provide several advantages, benefits, and/or improvements over existing sequencing systems, including SV callers and other sequencing data analysis software. For instance, the call refinement system generates more accurate structural variant calls than existing sequencing systems. While some prior sequencing systems inaccurately generate structural variant calls (especially for small size structural variants), the call refinement system trains or utilizes a structural variant refinement machine-learning model to improve structural variant calling over prior systems. Specifically, as mentioned, the call refinement system can correct truth datasets for training the structural variant refinement machine-learning model on more precise training data, thereby producing more accurate structural variant calls (and reducing false positives and/or false negatives). Further contributing to the improve accuracy in structural variant calling, the call refinement system determines and utilizes specific sequencing metrics (unique from prior systems) as a basis for generating calls (e.g., as input data) via the structural variant refinement machine-learning model.

To accomplish the aforementioned improved accuracies, as indicated, the call refinement system utilizes an improved and unique machine-learning model—the structural variant refinement machine-learning model—that is trained to perform new applications. Unlike existing variant callers that generate nucleotide base calls from general sequencing data—without adjustment or emphasis on whether a particular genomic coordinate historically exhibits or has been detected to exhibit a structural variant—the call refinement system utilizes a unique structural variant refinement machine-learning model that generates specific variant call classifications for structural variants. In some cases, the call refinement system utilizes the structural variant refinement machine-learning model as a post processing filter to update a structural variant call generated by a call generation model from the same (or a subset of the same) sequencing metrics used by the structural variant refinement machine-learning model.

In addition to improved accuracy, in certain embodiments, the call refinement system improves computing efficiency and speed. As noted above, some existing sequencing systems utilize computationally expensive, slow neural network architectures (e.g., deep learning architectures such as convolutional neural networks) that require many hours (e.g., 5-8 hours to analyze base-call data for a genomic sample with multiple processors executing on a server) and large amounts of computational resources to implement and generate variant calls from a sequencing run. Such deep learning architectures can further require several days (or weeks) to train. Conversely, the call refinement system utilizes a comparatively lightweight, fast architecture for the structural variant refinement machine-learning model. In contrast to the many hours across multiple processors required by existing sequencing systems, the call refinement system requires under an hour (for both the call generation model and the structural variant refinement machine-learning model together) of runtime on a single field programmable gate array or a single processor to generate structural variant calls for a genomic sample. Thus, the call refinement system is far faster and less computationally expensive than many deep learning approaches to variant calling. Not only are the models of the call refinement system faster and less computationally expensive to implement, but the structural variant refinement machine-learning model is also much faster and less computationally expensive to train than many existing deep learning systems.

Additionally, the machine learning architecture of the call refinement system can be trained using much less training data than the deep learning architectures of prior systems. Such computationally lighter training is especially important for structural variant calling as the number of structural variants in a given genomic sample is relatively small—much smaller than the number of single nucleotide variants (or other variant types). Thus, even for the limited amount of data for structural variant calling, the call refinement system can converge on accurate predictions, unlike prior systems that require much more data and struggle to generate accurate predictions for structural variants.

As a further advantage over existing sequencing systems, in certain implementations, the call refinement system can identify or facilitate changes to individual sequencing metrics that affect the accuracy of structural variant calls. While neural network architectures of many existing sequencing systems render interpretation of internal model data impossible with hidden, latent features among their many layers and neurons, the call refinement system utilizes model architectures that facilitate interpretation of the effect of individual sequencing metrics. More specifically, in some cases, the call refinement system utilizes a call generation model and a structural variant refinement machine-learning model (e.g., gradient boosted trees, random-forest model) that enable extraction and analysis of individual sequencing metrics used throughout the process of generating a structural variant call. Indeed, the call refinement system can determine respective importance measures for sequencing metrics involved in determining a structural variant call at a particular region of genomic coordinates.

As suggested by the foregoing discussion, this disclosure utilizes a variety of terms to describe features and benefits of the call refinement system. Additional detail is hereafter provided regarding the meaning of these terms as used in this disclosure. As used in this disclosure, for instance, the term “genomic sequence” or “sample sequence” refers to a sequence of nucleotides isolated or extracted from a sample organism (or a copy of such an isolated or extracted sequence). In particular, a genomic sequence includes a segment of a nucleic acid polymer that is isolated or extracted from a sample organism and composed of nitrogenous heterocyclic bases. For example, a genomic sequence can include a segment of deoxyribonucleic acid (DNA), ribonucleic acid (RNA), or other polymeric forms of nucleic acids or chimeric or hybrid forms of nucleic acids noted below. More specifically, in some cases, the genomic sequence is found in a sample prepared or isolated by a kit and received by a sequencing device.

Relatedly, as used herein, the term “genomic sample” refers to a target genome or portion of a genome undergoing an assay or sequencing. For example, a genomic sample includes one or more sequences of nucleotides isolated or extracted from a sample organism (or a copy of such an isolated or extracted sequence). In particular, a genomic sample includes a full genome that is isolated or extracted (in whole or in part) from a sample organism and composed of nitrogenous heterocyclic bases. A genomic sample can include a segment of deoxyribonucleic acid (DNA), ribonucleic acid (RNA), or other polymeric forms of nucleic acids or chimeric or hybrid forms of nucleic acids noted below. In some cases, the genomic sample is found in a sample prepared or isolated by a kit and received by a sequencing device.

As further used herein, the term “structural variant” refers to a variation (e.g., deletion, insertion, translocation, inversion) in a structure of an organism's chromosome or a variation to the nucleotide sequences of the organism's chromosome. In some cases, a structural variant includes a variation to a threshold number of base pairs (e.g., >50 base pairs) within an organism's chromosome. Accordingly, in certain implementations, a structural variant includes an insertion or deletion exceeding a threshold number of base pairs, a duplication exceeding a threshold number of base pairs, an inversion, a translocation, or a copy number variation (CNV). While this disclosure describes some examples of 50 base pairs as a threshold number of base pairs, in some embodiments, the threshold number of base pairs for a structural variant may be different, such as 35, 45, 100, or 1,000 base pairs.

Relatedly, the term “small size structural variant” refers to a structural variant having a size or a length of less than a threshold number (e.g., 200, 300, 500 or some other threshold) of base pairs. For example, a small size structural variant can include a structural variant within a window or a size range of between 50 and 200 base pairs (or within some other window with different upper and lower thresholds, such as 100 to 200 base pairs). Along these lines, the term “structural variant call” (e.g., a “small size structural variant call”) refers to a determination or prediction of a structural variant for one or more genomic coordinates of a genomic sample. For example, a structural variant call can be predicted or determined by one or more sequencing processes, via a call generation model, and/or utilizing a structural variant refinement machine-learning model.

Additionally, as used herein, the term “nucleotide read” refers to an inferred sequence of one or more nucleotide bases (or nucleotide base pairs) from all or part of a sample nucleotide sequence (e.g., a sample genomic sequence, cDNA). In particular, a nucleotide read includes a determined or predicted sequence of nucleotide base calls for a nucleotide fragment (or group of monoclonal nucleotide fragments) from a sample library fragment corresponding to a genomic sample. For example, a sequencing device determines a nucleotide read by generating nucleotide base calls for nucleotide bases passed through a nanopore of a nucleotide-sample slide, determined via fluorescent tagging, or determined from a well in a flow cell.

As noted above, in some embodiments, the call refinement system determines sequencing metrics for generating structural variant calls. As used herein, the term “sequencing metric” refers to a quantitative measurement or score indicating a degree to which one or more nucleotide base calls (e.g., predictions of nucleotide bases at respective genomic coordinates) align, compare, or quantify with respect to a genomic coordinate or a genomic region of a reference genome, with respect to nucleotide base calls from nucleotide reads, or with respect to external genomic sequencing or genomic structure. For instance, a sequencing metric includes a quantitative measurement or score indicating a degree to which (i) individual nucleotide base calls from a nucleotide read align, map, or cover a genomic coordinate or reference base of a reference genome; (ii) nucleotide base calls compare to reference or alternative nucleotide reads in terms of mapping, mismatch, base call quality, or other raw sequencing metrics; or (iii) genomic coordinates or regions corresponding to nucleotide base calls demonstrate mappability, repetitive base call content, DNA structure, or other generalized metric. In some embodiments, a sequencing metric is an input to a machine-learning model from which the machine-learning model can generate predictions for nucleotide base calls, including structural variant calls. Indeed, any of the sequencing metrics described herein may be an input for a structural variant refinement machine-learning model.

Indeed, in certain embodiments, sequencing metrics can be grouped into different sequencing metric categories for quantitative measurements, including: (i) “read-based sequencing metrics” that are derived from nucleotide reads and that indicate a degree to which nucleotide base calls from a nucleotide read (or one or more nucleotide reads) compare to reference or alternative nucleotide bases in terms of mapping, mismatch, base call quality, or other raw sequencing metrics; (ii) “variant region quality sequencing metrics” that indicate a degree to which nucleotide base calls satisfy read quality thresholds (e.g., derive from a nucleotide read comprising a threshold number of base calls) or base call quality thresholds (e.g., threshold Q score) at a genomic coordinate or region corresponding to a structural variant; or (iii) “reference-based sequencing metrics” indicating a degree to which genomic coordinates or regions corresponding to nucleotide base calls demonstrate mappability, repetitive base call content (e.g., guanin quadruplex), permutation entropy, DNA structure, or other generalized metrics.

In some cases, variant region quality sequencing metrics refer to specific scores or other measurements indicating an accuracy of a nucleotide base call. In particular, a “base call quality metric” comprises a value indicating a likelihood that one or more predicted nucleotide base calls for a genomic coordinate contain errors. For example, in certain implementations, a base call quality metric can comprise a Q score (e.g., a Phred quality score) predicting the error probability of any given nucleotide base call. To illustrate, a quality score (or Q score) may indicate that a probability of an incorrect nucleotide base call at a genomic coordinate is equal to 1 in 100 for a Q20 score, 1 in 1,000 for a Q30 score, 1 in 10,000 for a Q40 score, etc.

Relatedly, in some embodiments, the call refinement system can generate sequencing metrics through modification or updating of previous metrics, such as re-engineered sequencing metrics. Indeed, as used herein, the term “re-engineered sequencing metrics” refers to sequencing metrics that have been updated, modified, augmented, refined, or re-engineered to measure or compare nucleotide base calls (e.g., nucleotide base calls for reads or variant calls) with respect to other nucleotide base calls, a standard or reference, or for targeted for a particular objective or task. For example, re-engineered sequencing metrics can include modifications to, or combinations of, raw sequencing metrics. In some embodiments, for instance, the call refinement system generates one or more of the read-based sequencing metrics, the reference-based sequencing metrics, and/or the variant region quality sequencing metrics as re-engineered sequencing metrics. In some cases, re-engineered sequencing metrics refer to sequencing metrics that are generated by the call refinement system and are therefore proprietary or internal to the call refinement system and not available to third-party systems. Example re-engineered sequencing metrics include a comparative-mapping-quality-distribution metric indicating a comparison between mapping quality distributions associated with a reference sequence and alternate contiguous sequences or a comparative-base-quality metric indicating comparisons between base qualities of a reference sequence and alternate contiguous sequences.

As further used herein, the term “genomic coordinate” (or sometimes simply “coordinate”) refers to a particular location or position of a nucleotide base within a genome (e.g., an organism's genome or a reference genome). In some cases, a genomic coordinate includes an identifier for a particular chromosome of a genome and an identifier for a position of a nucleotide base within the particular chromosome. For instance, a genomic coordinate or coordinates may include a number, name, or other identifier for a chromosome (e.g., chr1 or chrX) and a particular position or positions, such as numbered positions following the identifier for a chromosome (e.g., chr1:1234570 or chr1:1234570-1234870). Further, in certain implementations, a genomic coordinate refers to a source of a reference genome (e.g., mt for a mitochondrial DNA reference genome or SARS-CoV-2 for a reference genome for the SARS-CoV-2 virus) and a position of a nucleotide base within the source for the reference genome (e.g., mt:16568 or SARS-CoV-2:29001). By contrast, in certain cases, a genomic coordinate refers to a position of a nucleotide base within a reference genome without reference to a chromosome or source (e.g., 29727).

As noted above, a genomic coordinate includes a position within a reference genome. Such a position may be within a particular reference genome. As used herein, the term “reference genome” refers to a digital nucleic acid sequence assembled as a representative example (or representative examples) of genes and other genetic sequences of an organism. Regardless of the sequence length, in some cases, a reference genome represents an example set of genes or a set of nucleic acid sequences in a digital nucleic acid sequenced determined by scientists as representative of an organism of a particular species. For example, a linear human reference genome may be GRCh38 or other versions of reference genomes from the Genome Reference Consortium. GRCh38 may include alternate contiguous sequences representing alternate haplotypes, such as SNPs and small indels (e.g., 10 or fewer base pairs, 50 or fewer base pairs). While GRCh38 may include alternate contiguous sequences representing alternate haplotypes, such as SNPs and small indels (e.g., 10 or fewer base pairs, 50 or fewer base pairs), GRCh38 includes alternate haplotypes with limited representation of population structural variants. Indeed, the structural variants represented in GRCh38 include only those represented by the 11 individuals whose libraries GRCh38 is constructed upon. As a further example, a reference genome may include a graph reference genome that includes both a linear reference genome and alternate contiguous sequences or other alternative paths representing nucleic acid sequences from ancestral haplotypes, such as Illumina DRAGEN Graph Reference Genome hg19.

Additionally, as used herein, the term “graph reference genome” refers to a reference genome that includes both a linear reference genome and alternate contiguous sequences (or graph augmentations) representing variant haplotype sequences or other variant or alternative nucleic-acid sequences. For instance, a graph reference genome can include a linear reference genome and alternate contiguous sequences corresponding to one or more population haplotype sequences identified from a genomic sample database. As an example, a graph reference genome may include the Illumina DRAGEN Graph Reference Genome hg19.

As further used herein, the term “contiguous sequence” (or “contig assembly”) refers to a consensus nucleotide sequence for a genomic region of a genomic sample (or multiple genomic samples of a species) based on a set of overlapping nucleotide segments corresponding to the genomic region. In particular, a contiguous sequence includes a consensus nucleotide sequence for a genomic region of one or more genomic samples based on nucleotide reads for the one or more genomic samples covering (or overlapping with) the genomic region. As noted above, the terms “contiguous sequence” and “contig assembly” can be used interchangeably.

Relatedly, the term “alternate contiguous sequence” (or simply “alt contig”) refers to a contiguous sequence representing a population haplotype added to a linear reference genome (or other reference genome) at a particular genomic coordinate or genomic coordinates (e.g., lifted over to the linear reference genome). In some implementations, a graph reference genome can include alternate contiguous sequences mapped to genomic coordinates of a primary assembly for a linear reference genome. For example, an alternate contiguous sequence may represent a population haplotype containing a structural variant with liftover to two or more genomic coordinates in the linear reference genome corresponding to two or more flanks of structural variant breakends. In some cases, a hash table for a graph reference genome includes identifiers that associate alternate contiguous sequences representing structural variant haplotypes with genomic coordinates representing reference haplotypes from a primary assembly for a linear reference genome.

As further used herein, the term “alignment score” refers to a numeric score, metric, or other quantitative measurement evaluating an accuracy of an alignment between a nucleotide read (or a fragment of the nucleotide read) and another nucleotide sequence from a reference genome. In particular, an alignment score includes a metric indicating a degree to which the nucleotide bases of a nucleotide read (or fragment of the nucleotide read) match or are similar to a reference sequence or an alternate contiguous sequence from a reference genome. In certain implementations, an alignment score takes the form of a Smith-Waterman score or a variation or version of a Smith-Waterman score for local alignment, such as various settings or configurations used by DRAGEN by Illumina, Inc. for Smith-Waterman scoring.

As suggested above, the call refinement system can utilize a machine-learning model to refine or update structural variant calls. As used herein, the term “machine-learning model” refers to a computer algorithm or a collection of computer algorithms that automatically improve for a particular task through experience based on use of data. For example, a machine-learning model can utilize one or more learning techniques to improve in accuracy and/or effectiveness. Example machine-learning models include various types of decision trees, support vector machines, Bayesian networks, or neural networks. In some cases, the structural variant refinement machine-learning model is a series of gradient boosted decision trees (e.g., XGBoost algorithm), while in other cases the structural variant refinement machine-learning model is a random forest model, a multilayer perceptron, a linear regression, a support vector machine, a deep tabular learning architecture, a deep learning transformer (e.g., self-attention-based-tabular transformer), or a logistic regression.

In some cases, the call refinement system utilizes a structural variant refinement machine-learning model to modify or update a structural variant call (e.g., a small size structural variant call) based on sequencing metrics. As used herein, the term “structural variant refinement machine-learning model” refers to a machine-learning model that generates structural variant call classifications. For example, in some cases, the structural variant refinement machine-learning model is trained to generate a false positive likelihood indicating a likelihood or a probability that a structural variant call is a false positive based on the sequencing metrics. In certain embodiments, a structural variant refinement machine-learning model includes multiple sub-models or operates in tandem with another structural variant refinement machine-learning model. As described further below, in some embodiments, a structural variant refinement machine-learning model generates, based on one or more sequencing metrics and/or an initial structural variant call, a likelihood score indicating a likelihood (e.g., a value between 0 to 1) indicating a likelihood that a particular structural variant is present at one or more genomic coordinates of a genomic sample. For instance, in certain implementations, the structural variant refinement machine-learning model generates, based on one or more sequencing metrics and/or an initial structural variant call as inputs, a likelihood score that is used as a posterior genotype likelihood (e.g., a PHRED-scaled-genotype likelihood) upon which a structural variant call is determined.

As mentioned, in some embodiments, the structural variant refinement machine-learning model can be a neural network. The term the term “neural network” refers to a machine-learning model that can be trained and/or tuned based on inputs to determine classifications or approximate unknown functions. For example, a neural network includes a model of interconnected artificial neurons (e.g., organized in layers) that communicate and learn to approximate complex functions and generate outputs (e.g., generated digital images) based on a plurality of inputs provided to the neural network. In some cases, a neural network refers to an algorithm (or set of algorithms) that implements deep learning techniques to model high-level abstractions in data. For example, a neural network can include a convolutional neural network, a recurrent neural network (e.g., an LSTM), a graph neural network, a self-attention transformer neural network, or a generative adversarial neural network.

As further used herein, the term “false positive likelihood” refers to a likelihood that a variant call is a false positive call. In particular, a false positive likelihood includes a likelihood (e.g., a value between 0 and 1) that an initial structural variant call determined by a call generation model is a false positive structural variant call. In some cases, a false positive likelihood could be represented as a likelihood score that an initial structural variant call (or a particular type or a particular length of structural variant call) is present or a false positive structural variant call. For example, in some embodiments, a false positive likelihood may be used as a posterior genotype likelihood (e.g., a PHRED-scaled-genotype likelihood) upon which a structural variant call is determined. Accordingly, in some embodiments, a structural variant refinement machine-learning model generates a likelihood score indicating a likelihood (e.g., a value between 0 to 1) indicating a likelihood that a particular structural variant is present at one or more genomic coordinates of a genomic sample. As indicated above, the term “structural variant false positive likelihood” may be used interchangeably in this disclosure with “false positive likelihood.” In some cases, a false positive likelihood includes a likelihood that an initial structural variant call is a false positive call versus a true positive call based on sequencing metrics.

As mentioned, in some embodiments, the call refinement system modifies data fields corresponding to a variant call file. As used herein, the term “variant call file” refers to a digital file that indicates or represents one or more nucleotide base calls (e.g., variant calls) compared to a reference genome along with other information pertaining to the nucleotide base calls (e.g., variant calls). For example, a variant call format (VCF) file refers to a text file format that contains information about variants at specific genomic coordinates, including meta-information lines, a header line, and data lines where each data line contains information about a single nucleotide base call (e.g., a single variant). As described further below, the call refinement system can generate different versions of variant call files, including a pre-filter variant call file comprising variant nucleotide base calls that either pass or fail a quality filter for base call quality metrics or a post-filter variant call file comprising variant nucleotide base calls that pass the quality filter but excludes variant nucleotide base calls that fail the quality filter.

As noted, in some embodiments, the call refinement system utilizes a call generation model to generate a nucleotide base call for a genomic coordinate. As used herein, the term “call generation model” refers to a probabilistic model that generates sequencing data from nucleotide reads of a genomic sequence, including nucleotide base calls, structural variant calls, and associated metrics. For example, in some cases, a call generation model refers to a Bayesian probability model that generates structural variant calls based on nucleotide reads of a genomic sequence. Such a model can process or analyze sequencing metrics corresponding to read pileups (e.g., multiple nucleotide reads corresponding to a single genomic coordinate), including mapping quality, base quality, and various hypotheses including foreign reads, missing reads, joint detection, and more. A call generation model may likewise include multiple components, including, but not limited to, different software applications or components for mapping and aligning, sorting, duplicate marking, computing read pileup depths, and variant calling. In some cases, the call generation model refers to the ILLUMINA DRAGEN model for structural variant calling functions and mapping and alignment functions.

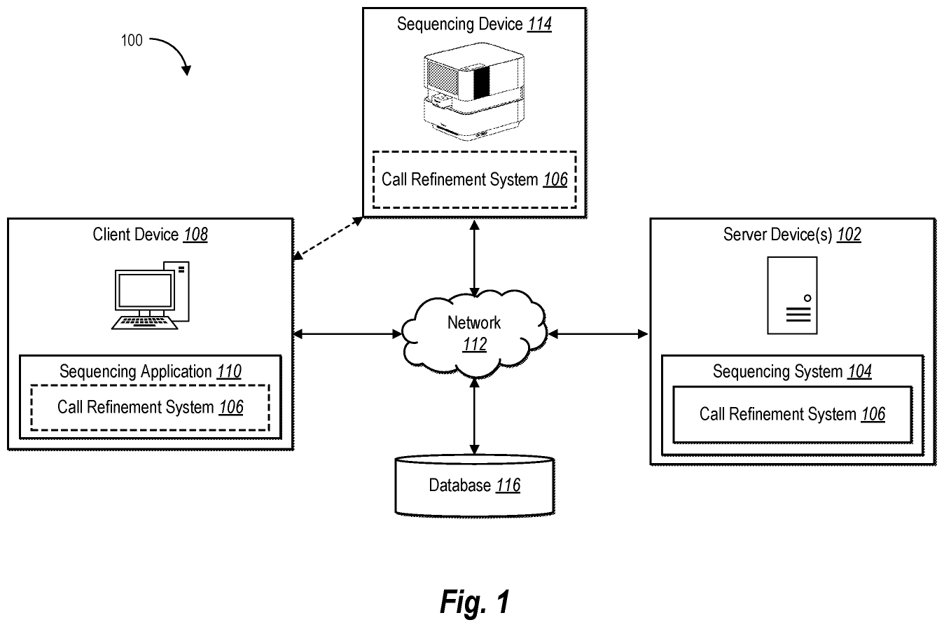

The following paragraphs describe the call refinement system with respect to illustrative figures that portray example embodiments and implementations. For example, FIG. 1 illustrates a schematic diagram of a computing system 100 in which a call refinement system 106 operates in accordance with one or more embodiments. As illustrated, the computing system 100 includes one or more server device(s) 102 connected to a client device 108 and a sequencing device 114 via a network 112. While FIG. 1 shows an embodiment of the call refinement system 106, this disclosure describes alternative embodiments and configurations below.

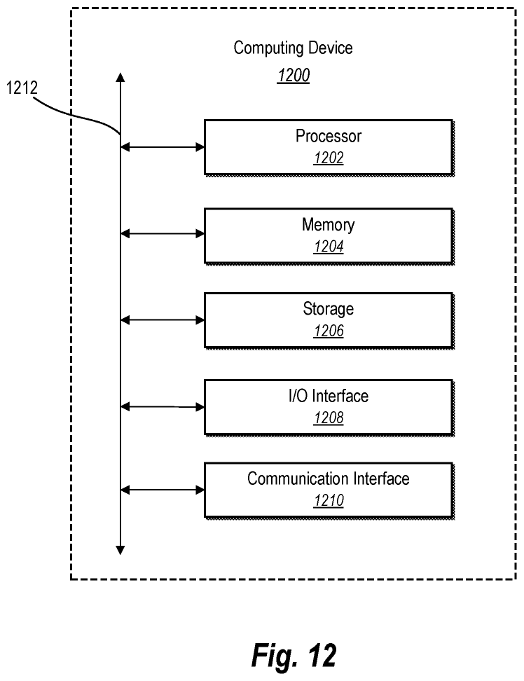

As shown in FIG. 1, the server device(s) 102, the client device 108, and the sequencing device 114 can communicate with each other via the network 112. The network 112 comprises any suitable network over which computing devices can communicate. Example networks are discussed in additional detail below with respect to FIG. 12.

As indicated by FIG. 1, the sequencing device 114 comprises a device for sequencing a nucleic acid polymer. In some embodiments, the sequencing device 114 analyzes nucleic acid segments or oligonucleotides extracted from genomic samples to generate nucleotide reads or other data utilizing computer implemented methods and systems (described herein) either directly or indirectly on the sequencing device 114. More particularly, the sequencing device 114 receives and analyzes, within nucleotide-sample slides (e.g., flow cells), nucleic acid sequences extracted from samples. In one or more embodiments, the sequencing device 114 utilizes SBS to sequence nucleic acid polymers into nucleotide reads. In addition or in the alternative to communicating across the network 112, in some embodiments, the sequencing device 114 bypasses the network 112 and communicates directly with the client device 108.

As further indicated by FIG. 1, the server device(s) 102 may generate, receive, analyze, store, and transmit digital data, such as data for determining base calls, structural variant calls, or sequencing nucleic acid polymers. As shown in FIG. 1, the sequencing device 114 may send (and the server device(s) 102 may receive) call data from the sequencing device 114. The server device(s) 102 may also communicate with the client device 108. In particular, the server device(s) 102 can send data to the client device 108, including a variant call file or other information indicating nucleotide base calls (e.g., structural variant calls or other variant calls), sequencing metrics, error data, or other metrics.

In some embodiments, the server device(s) 102 comprise a distributed collection of servers where the server device(s) 102 include a number of server devices distributed across the network 112 and located in the same or different physical locations. Further, the server device(s) 102 can comprise a content server, an application server, a communication server, a web-hosting server, or another type of server. In some cases, the server device(s) 102 are located at a same physical location as the sequencing device 114.

As further shown in FIG. 1, the server device(s) 102 can include a sequencing system 104. Generally, the sequencing system 104 analyzes call data, such as nucleotide base calls for nucleotide reads and sequencing metrics received from the sequencing device 114, to determine nucleotide base sequences for nucleic acid polymers. For example, the sequencing system 104 can receive raw data from the sequencing device 114 and can determine a consensus nucleotide base sequence for a segment of a genomic sample aligned with a reference genome. In some embodiments, the sequencing system 104 determines the sequences of nucleotide bases in DNA and/or RNA segments or oligonucleotides. In addition to processing and determining sequences for nucleic acid polymers, the sequencing system 104 also generates a variant call file indicating one or more nucleotide base calls and/or structural variant calls for one or more genomic coordinates or regions.

As just mentioned, and as illustrated in FIG. 1, the call refinement system 106 analyzes call data, such as sequencing metrics from the sequencing device 114, to determine structural variant calls for one or more genomic samples. In some cases, the call refinement system 106 includes a call generation model and a structural variant refinement machine-learning model. In some embodiments, the call refinement system 106 determines sequencing metrics for genomic sequences. Based on data derived or prepared from the sequencing metrics, the call refinement system 106 applies a call generation model to determine initial structural variant calls for the sample sequence corresponding to genomic coordinates. The call refinement system 106 further utilizes a structural variant refinement machine-learning model to generate modified/refined/updated structural variant calls corresponding to the initial structural variant calls. Based on such data, for example, the call refinement system 106 can update data fields corresponding to a variant call file to confirm or modify a structural variant call for improved accuracy.

As further illustrated and indicated in FIG. 1, the client device 108 can generate, store, receive, and send digital data. In particular, the client device 108 can receive sequencing metrics from the sequencing device 114. Furthermore, the client device 108 may communicate with the server device(s) 102 to receive a variant call file comprising structural variant calls and/or other metrics, such as a base-call quality scores, coverage depth, a genotype indication, and/or a genotype quality. The client device 108 can accordingly present or display information pertaining to the structural variant call within a graphical user interface to a user associated with the client device 108. For example, the client device 108 can present an importance measure interface that includes a visualization or a depiction of various importance measures associated with, or attributed to, individual sequencing metrics with respect to a particular structural variant call.

The client device 108 illustrated in FIG. 1 may comprise various types of client devices. For example, in some embodiments, the client device 108 includes non-mobile devices, such as desktop computers or servers, or other types of client devices. In yet other embodiments, the client device 108 includes mobile devices, such as laptops, tablets, mobile telephones, or smartphones. Additional details regarding the client device 108 are discussed below with respect to FIG. 12.

As further illustrated in FIG. 1, the client device 108 includes a sequencing application 110. The sequencing application 110 may be a web application or a native application stored and executed on the client device 108 (e.g., a mobile application, desktop application). The sequencing application 110 can include instructions that (when executed) cause the client device 108 to receive data from the call refinement system 106 and present, for display at the client device 108, data from a variant call file. Furthermore, the sequencing application 110 can instruct the client device 108 to display a visualization of importance measures for sequencing metrics of a structural variant call.

As further illustrated in FIG. 1, the call refinement system 106 may be located on the client device 108 as part of the sequencing application 110 or on the sequencing device 114. Accordingly, in some embodiments, the call refinement system 106 is implemented by (e.g., located entirely or in part) on the client device 108. In yet other embodiments, the call refinement system 106 is implemented by one or more other components of the computing system 100, such as the sequencing device 114. In particular, the call refinement system 106 can be implemented in a variety of different ways across the server device(s) 102, the network 112, the client device 108, and the sequencing device 114. For example, the call refinement system 106 can be downloaded from the server device(s) 102 to the client device 108 and/or to the sequencing device 114 where all or part of the functionality of the call refinement system 106 is performed at each respective device within the computing system 100.

As further illustrated in FIG. 1, the computing system 100 includes a database 116. The database 116 can store information, such as variant call files, genomic sequences, nucleotide reads, nucleotide base calls, structural variant calls, and sequencing metrics. In some embodiments, the server device(s) 102, the client device 108, and/or the sequencing device 114 communicate with the database 116 (e.g., via the network 112) to store and/or access information, such as variant call files, genomic sequences, nucleotide reads, nucleotide base calls, structural variant calls, and sequencing metrics. In some cases, the database 116 also stores one or more models, such as a structural variant refinement machine-learning model and/or a call generation model.

Though FIG. 1 illustrates the components of computing system 100 communicating via the network 112, in certain implementations, the components of computing system 100 can also communicate directly with each other, bypassing the network 112. For instance, and as previously mentioned, in some implementations, the client device 108 communicates directly with the sequencing device 114. Additionally, in some embodiments, the client device 108 communicates directly with the call refinement system 106. Moreover, the call refinement system 106 can access one or more databases housed on or accessed by the server device(s) 102 or elsewhere in the computing system 100.

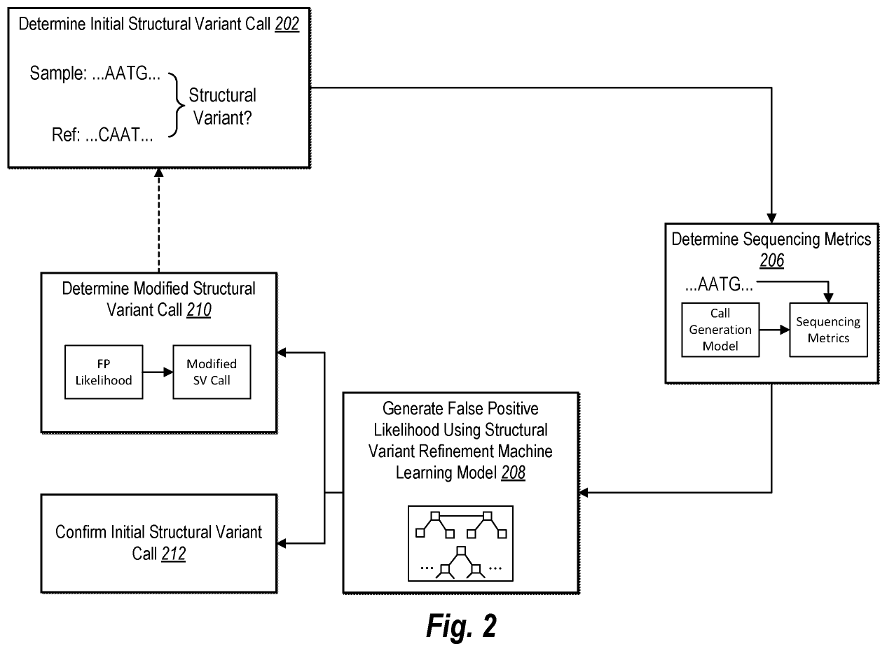

As indicated above, the call refinement system 106 can confirm an initial structural variant call or determine a modified structural variant call utilizing a structural variant refinement machine-learning model. In particular, the call refinement system 106 can generate an initial structural variant call utilizing a call generation model and can confirm or refine the initial structural variant call with a structural variant refinement machine-learning model trained specifically to reduce (e.g., minimize) false positives and false negatives based on certain sequencing metrics. FIG. 2 illustrates an example sequence of acts for determining a modified structural variant call or confirming the initial structural variant call utilizing a structural variant refinement machine-learning model in accordance with one or more embodiments. The description of FIG. 2 provides an overview of generating a modified structural variant call or confirming the initial structural variant call, and additional detail regarding the various acts is provided thereafter with reference to subsequent figures.

As illustrated in FIG. 2, the call refinement system 106 can perform an act 202 to determine initial structural variant call. In particular, the call refinement system 106 determines an initial structural variant call utilizing a call generation model. For example, the call refinement system 106 call utilizes the call generation model to process or analyze sequencing metrics to determine a structural variant call at one or more genomic coordinates of a genomic sample. For example, the call refinement system 106 applies a number of Bayesian probabilistic models or algorithms to derive various probabilities for different nucleotide bases, quality metrics, mapping metrics, joint metrics, and other data occurring within nucleotide reads for a genomic sample.

By utilizing the probabilistic model, the call refinement system 106 determines a structural variant call that indicates a predicted structural variation for the genomic sample at one or more genomic coordinates as compared to a reference genome. For instance, the call refinement system 106 determines the initial structural variant call by determining one or more of: i) a deletion of more than a threshold number of base pairs, ii) an insertion of more than the threshold number of base pairs, iii) a duplication of more than the threshold number of base pairs, iv) an inversion, v) a translocation, vi) or a copy number variation (CNV). The call refinement system 106 can utilize the call generation model to generate a plurality of structural variant calls for different genomic coordinates or regions of the genomic sample in comparison with a reference genome.

In addition to determining an initial structural variant call, the call refinement system 106 can perform an act 206 to determine sequencing metrics. More specifically, the call refinement system 106 can determine sequencing metrics from sequencing data associated with nucleotide reads of a genomic sample, from reference data associated with a reference genome, and/or from call data associated with structural variant calls (e.g., small size structural variant calls). For example, the call refinement system 106 determines sequencing metrics based on initial sequencing data from a sequencing device (e.g., the sequencing device 114) and/or based on call data from a call generation model.

In some embodiments, the call refinement system 106 determines different types of sequencing metrics, including reference-based sequencing metrics, read-based sequencing metrics, and variant region quality sequencing metrics. In some cases, the call refinement system 106 determines reference-based sequencing metrics by analyzing genomic regions of a reference genome corresponding to genomic coordinates (e.g., an SV region used as the basis for making a structural variant call) of a genomic sample. Such reference-based sequencing metrics can include, but are not limited to: i) a tandem repeat length in nucleotide bases, ii) a permutation entropy of nucleotide bases, iii) presence of a cytosine quadruplex (C-quadruplex), and/or iv) presence of a guanine quadruplex (G-quadruplex). Additional detail regarding the various reference-based sequencing metrics is provided below with reference to subsequent figures.

As noted above, the call refinement system 106 can also determine read-based sequencing metrics. For example, the call refinement system 106 can utilize a sequencing device (e.g., the sequencing device 114) and/or a call generation model to determine read data associated with a genomic sample. In some cases, the call refinement system 106 utilizes the call generation model to determine an initial structural variant call for a genomic region of a genomic sample and to further determine one or more sequencing metrics associated with the initial structural variant call. Such read-based sequencing metrics can include, but are not limited to: i) one or more base-call quality scores, ii) a fraction of nucleotide reads supporting an alternate contiguous sequence from a reference genome, iii) a number of split nucleotide reads from nucleotide reads corresponding to an initial structural variant call, iv) a coverage depth of nucleotide reads corresponding to an initial structural variant call, v) an additional structural variant call located within a threshold number of base pairs from an initial structural variant call within a genomic sample, vi) an alignment of a contiguous sequence corresponding to nucleotide reads with a reference sequence of a reference genome modified to include a structural variant corresponding to the initial structural variant call, vii) a deletion length in nucleotide bases based on one or more soft clipped nucleotide reads, viii) a number of the nucleotide reads that exhibit a mapping quality metric that fails to satisfy a threshold mapping quality metric, ix) an insert size representing a length of nucleotide-read fragments corresponding to the initial structural variant call, and/or x) a structural-variant likelihood representing a ratio of the initial structural variant call to a reference call for the one or more genomic coordinates based on an insert size.

Additionally, in certain embodiments, the call refinement system 106 determines variant region quality sequencing metrics. For example, the call refinement system 106 can utilize a sequencing device (e.g., the sequencing device 114) and/or a call generation model to determine variant region quality sequencing metrics associated with genomic coordinates of genomic sample and/or associated with an initial structural variant call. In some cases, the call refinement system 106 determines variant region quality sequencing metrics by determining information associated with predicted nucleotide base calls and/or structural variant calls (e.g., as generated by the sequencing device 114 and/or the call generation model). Such variant region quality sequencing metrics can include, but are not limited to: i) a number of nucleotide reads that comprise at least a threshold number of base calls and correspond to a target genomic region for the initial structural variant call and/or ii) a number of nucleotide bases in an alternate contiguous sequence from a reference genome for which based calls for nucleotide reads fail to satisfy a threshold base call quality score.

As also illustrated in FIG. 2, in one or more embodiments, the call refinement system 106 performs an act 208 to generate a false positive likelihood using a structural variant refinement machine-learning model. In particular, the call refinement system 106 utilizes the structural variant refinement machine-learning model to generate or predict a false positive likelihood based on one or more sequencing metrics, including read-based sequencing metrics, reference-based sequencing metrics, and variant region quality sequencing metrics. For instance, in some embodiments, the structural variant refinement machine-learning model uses a series of gradient boosted trees to process or analyze the sequencing metrics according to various internal weights or parameters to ultimately generate a false positive likelihood indicating a likelihood that the initial structural variant call (as determined via the act 202) is a false positive. In some cases, the call refinement system 106 also trains the structural variant refinement machine-learning model by adjusting one or more of its parameters according to training data generated by correcting errors in a truth dataset. Additional detail regarding training an implementing the structural variant refinement machine-learning model for determining a false positive likelihood is provided below with reference to subsequent figures.

As further illustrated in FIG. 2, in one or more implementations, the call refinement system 106 performs an act 210 to determine a modified structural variant call. In particular, the call refinement system 106 determines a modified structural variant call based on the false positive likelihood determined via the act 208. For example, the call refinement system 106 examines candidate loci (e.g., candidate genomic coordinates, candidate genomic regions) for potential structural variants generated by a call generation model that were dropped or not called in a VCF (e.g., based on a threshold base-call-quality score, a threshold mapping quality metric, or some other or additional filtering criteria). The call refinement system 106 determines a false positive likelihood that functions as a likelihood score indicating whether a candidate locus (e.g., a locus that was indicated as a potential structural variant but that was ultimately indicated by a call generation model as not reflecting a structural variant) should be called a structural variant. In cases where a candidate locus is called as a structural variant, the call refinement system 106 corrects a false negative call to a true positive structural variant call.

Additionally or alternatively, in some embodiments, based on the false positive likelihood satisfying at least a threshold likelihood that an initial structural variant call is a false positive, the call refinement system 106 (i) modifies or corrects a positive structural variant call identifying a presence of a structural variant to a different variant call or reference call or (ii) modifies or corrects a negative structural variant call identifying an absence of a structural variant to a positive structural variant call or reference call. Indeed, in some cases, the call refinement system 106 also (or alternatively) determines a false negative likelihood (via the structural variant refinement machine-learning model) indicating a likelihood that an initial structural variant call is a false negative. The call refinement system 106 can further determine a modified structural variant call based on the false negative likelihood.

As an example of determining a modified structural variant call, the call refinement system 106 determines a structural variant call for one or more genomic coordinates (e.g., chr1:49263256) reflecting a deletion by identifying a single G in the sample nucleotide sequence where GTAAC exists in the reference sequence. As a further example, the call refinement system 106 determines a structural variant call that represents an insertion at a set genomic coordinates (e.g., chr1:7602080) by identifying a sequence of at least 50 base pairs (or some other threshold number of base pairs) but no more than 200 base pairs (or some other threshold number of base pairs) in the genomic sample where no such sequence exists in the reference genome.

As further shown in FIG. 2, in the alternative to determining a modified structural variant call, in some embodiments, the call refinement system 106 performs an act 212 of confirming the initial structural variant call. When a false positive likelihood from the structural variant refinement machine-learning model falls below a threshold (e.g., below 0.50), for instance, the call refinement system 106 determines that an initial structural variant call from a call generation model is correct. Based on the false positive likelihood failing to satisfy at least a threshold likelihood that an initial structural variant call is a false positive, for instance the call refinement system 106 (i) confirms a positive structural variant call identifying a presence of a structural variant or (ii) confirms a negative structural variant call identifying an absence of a structural variant. In some cases, as suggested above, the call refinement system 106 confirms a negative structural variant call for a candidate locus (e.g., candidate genomic coordinates, candidate genomic regions) for which a call generation model initially generated a candidate structural variant—or identified a potential structural variant—but ultimately determined that the candidate locus did not comprise a structural variant. Based on a false positive likelihood, from a structural variant refinement machine-learning model, failing to satisfy at least a threshold likelihood that an initial structural variant call is a false positive, the call refinement system 106 confirms a true negative structural variant call.

In one or more implementations, the call refinement system 106 generates the false positive likelihood (e.g., via the act 208) and/or determines the modified structural variant call (e.g., via the act 210) or confirms the initial structural variant call (e.g., via the act 212) while, or during the process of, determining the initial structural variant call (e.g., via the act 202). For example, the call refinement system 106 simultaneously or parallelly implements the structural variant refinement machine-learning model and the call generation model to generate an initial structural variant call and a false positive likelihood for modifying the initial structural variant call (e.g., based on one or more common sequencing metrics).

In some embodiments, the call refinement system 106 further modifies data fields corresponding to a variant call file of the initial structural variant call to generate a finalized or modified structural variant call (e.g., within a pre-filter or post-filter variant call file). Indeed, the call refinement system 106 generates the finalized (e.g., refined) structural variant call based on the false positive likelihood determined from some or all of the sequencing metrics processed by the call generation model (e.g., one or more of the same sequencing metrics used to generate the initial structural variant call). This simultaneous or parallel operation affords the call refinement system 106 improved computational efficiency and increased speed by recalibrating nucleotide base calls as they are initially generated (rather than performing one operation before the other).

As further shown, the call refinement system 106 can repeat the process illustrated in FIG. 2 for different genomic coordinates. For example, the call refinement system 106 can determine multiple initial structural variant calls at various genomic coordinates or genomic regions of a genomic sample. The call refinement system 106 can further determine sequencing metrics corresponding to initial structural variant calls for different genomic coordinate(s), generate false positive likelihoods, and determine modified structural variant calls for the genomic coordinate(s) of the genomic sample (e.g., to correct one or more initial variant calls at various genomic coordinates or SV regions) or confirm the initial structural variant calls for the genomic coordinate(s).

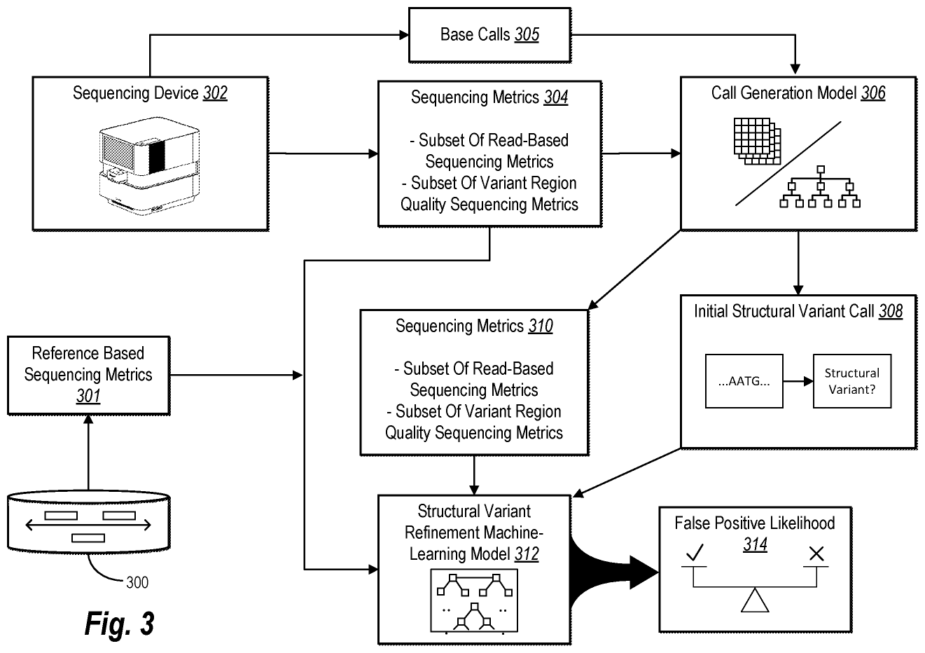

As mentioned above, in certain described embodiments, the call refinement system 106 determines a false positive likelihood using a structural variant refinement machine-learning model. In particular, the call refinement system 106 utilizes a structural variant refinement machine-learning model to generate, determine, or predict a false positive likelihood based on sequencing metrics associated with one or more genomic coordinates, such as an SV region of a genomic sample. FIG. 3 illustrates an example diagram of the call refinement system 106 generating a false positive likelihood utilizing a structural variant refinement machine-learning model in accordance with one or more embodiments.

As illustrated in FIG. 3, the call refinement system 106 utilizes a sequencing device 302 (e.g., the sequencing device 114) to determine base calls 305 for nucleotide reads for a genomic sample and sequencing metrics 304 corresponding to the base calls 305. For instance, the call refinement system 106 determines a subset of read-based sequencing metrics based on the nucleotide reads comprising the base calls 305. As indicated above, the subset of read-based sequencing metrics may include base-call quality scores for the base calls 305 or other sequencing metrics that are part of a base call (BCL) file generated by the sequencing device 302. In some cases, the call refinement system 106 further determines (or derives) a subset of variant region quality sequencing metrics from read data determined via the sequencing device 302. For instance, the subset of variant region quality sequencing metrics may include a count or number of nucleotide reads that include at least a threshold number of base calls and cover a target genomic region for a structural variant (e.g., a known structural variant satisfying a particular allele frequency).

As further shown in FIG. 3, the call refinement system 106 further utilizes the call generation model 306 to determine an initial structural variant call 308. Indeed, the call refinement system 106 utilizes the call generation model 306 to generate predictions for structural variants within a genomic sample based on the sequencing metrics 304 and/or other data from the sequencing device 302. The initial structural variant call 308 may include a positive structural variant call identifying a presence of a structural variant or a negative structural variant call identifying an absence of the structural variant. From the initial structural variant call 308 (and/or from other data associated with the call generation model 306), the call refinement system 106 further determines sequencing metrics 310, such as a subset of read-based sequencing metrics and a subset of variant region quality sequencing metrics.

To determine read-based sequencing metrics, the call refinement system 106 accesses, retrieves, obtains, determines, or generates nucleotide reads using the sequencing device 302. In particular, the call refinement system 106 determines nucleotide reads comprising nucleotide base calls for regions from a genomic sample (e.g., a sample nucleotide sequence). For example, the call refinement system 106 generates a plurality of nucleotide reads utilizing sequencing-by-synthesis (SBS) techniques and/or Sanger sequencing techniques to determine nucleotide base calls for oligonucleotide clusters from wells in a flow cell and/or via fluorescent tagging. More specifically, the call refinement system 106 utilizes cluster generation and SBS chemistry to sequence millions or billions of clusters in a flow cell. During SBS chemistry, for each cluster, the call refinement system 106 stores nucleotide base calls from nucleotide reads for every cycle of sequencing via real-time analysis (RTA) software.

In some embodiments, as part of determining the sequencing metrics 304, the call refinement system 106 performs read processing and mapping. For example, the call refinement system 106 utilizes RTA software to store base call data in the form of individual base call files (or BCLs). In some cases, the call refinement system 106 further converts the BCL files into sequence data (e.g., via BCL to FASTQ conversion). Additionally, the call refinement system 106 identifies multiple-read coverage (e.g., read pileups) that include multiple nucleotide reads or nucleotide base calls corresponding to a single genomic coordinate or genomic region (or a single SV region).

In particular, in certain embodiments, the call refinement system 106 aligns nucleotide reads with a reference genome or receives information pertaining to the read alignment. Specifically, the call refinement system 106 determines which nucleotide base(s) of a given nucleotide read align with which genomic coordinate of a reference sequence (or receives information indicating alignment). Different nucleotide reads have different lengths and include different nucleotide bases. Accordingly, in some cases, the call refinement system 106 analyzes each nucleotide of each read to determine (or receives information indicating) where the read “fits” in relation to a reference genome (or other reference sequence)—such as, where the bases within the read align with bases in the reference genome.

In certain embodiments, the call refinement system 106 performs additional statistical tests to determine or detect differences between sequencing metrics associated with a reference genome and sequencing metrics associated with alternate contiguous sequences. Through these statistical tests, the call refinement system 106 re-engineers raw sequencing metrics to determine read-based sequencing metrics. In some cases, the call refinement system 106 determines or extracts raw sequencing metrics that include one or more of (i) alignment metrics for quantifying alignment of nucleotide reads (of genomic samples) with genomic coordinates of a reference genome or another example nucleotide sequence (e.g., a nucleotide sequence from an ancestral haplotype), (ii) depth metrics for quantifying depth of nucleotide base calls for nucleotide reads at genomic coordinates of the reference genome, or (iii) call-quality metrics for quantifying quality of nucleotide base calls for nucleotide reads at genomic coordinates of the reference genome.

### A. Read-Based Sequencing Metrics

As part of read-based sequencing metrics, for instance, the call refinement system 106 determines mapping-quality metrics (e.g., the MAPQ metrics), soft-clipping metrics, or other alignment metrics that measure an alignment of nucleotide reads with a reference genome. In some embodiments, the call refinement system 106 determines the following read-based sequencing metrics: i) one or more base-call quality scores, ii) a fraction of nucleotide reads supporting an alternate contiguous sequence from a reference genome, iii) a number of split nucleotide reads from nucleotide reads corresponding to an initial structural variant call, iv) a coverage depth of nucleotide reads corresponding to an initial structural variant call, v) an additional structural variant call located within a threshold number of base pairs from an initial structural variant call within a genomic sample, vi) an alignment of a contiguous sequence corresponding to nucleotide reads with a reference sequence of a reference genome modified to include a structural variant corresponding to the initial structural variant call, vii) a deletion length in nucleotide bases based on one or more soft clipped nucleotide reads, viii) a number of the nucleotide reads that exhibit a mapping quality metric that fails to satisfy a threshold mapping quality metric, ix) an insert size representing a length of nucleotide-read fragments corresponding to the initial structural variant call (e.g., genomic coordinates of an SV region).

As just mentioned, in some embodiments, the call refinement system 106 re-engineers certain raw sequencing metrics to generate read-based sequencing metrics that are more informative for comparing metrics associated with a reference genome with sequencing metrics associated with various supporting alternate contiguous sequences. For example, the call refinement system 106 determines various metrics for a genomic sample in relation to a reference genome and further determines various metrics for the genomic sample in relation to alternate contiguous sequences. In addition, in some embodiments, the call refinement system 106 performs comparative analyses between metrics associated with the reference genome and the metrics associated with the alternate supporting reads of alternate contiguous sequences.

For instance, the call refinement system 106 compares how nucleotide bases of a nucleotide read map to a reference sequence (e.g., a reference genome) with how the nucleotide bases map to various alternate contiguous sequences. In particular, in some cases, the call refinement system 106 determines mapping qualities (e.g., MAPQ scores) of nucleotide reads mapped to a primary assembly of a reference genome to compare with mapping qualities (e.g., MAPQ scores) of the nucleotide reads mapped to alternative contiguous sequences. For example, the call refinement system 106 determines mapping quality statistics reflecting differences in the distribution of reads supporting a primary assembly versus reads supporting alternate contiguous sequences.

The following paragraphs describe read-based sequencing metrics i)-x) noted above in more detail along with associated metrics. As noted above, in these or other cases, the call refinement system 106 determines a base-call quality score for base calls within a nucleotide read. Specifically, the call refinement system 106 determines probabilities of correctness for nucleotide base calls of nucleotide reads (e.g., Phred+33 encoded). In some cases, the call refinement system 106 determines one or more base-call quality scores in the form of a DRAGEN QUAL score or a Q score for one or more nucleotide base calls. Further, the call refinement system 106 determines a fraction of nucleotide reads supporting an alternate contiguous sequence from a reference genome. For instance, the call refinement system 106 determines numbers of nucleotide reads supporting (e.g., matching or aligning with) an alternate contiguous sequence of a reference genome and numbers of nucleotide reads supporting a primary assembly within the reference genome. The call refinement system 106 further compares the aforementioned numbers and determines a fraction to reflect the comparison.

In some cases, the call refinement system 106 utilizes specific features to determine the fraction of reads supporting an alternate contiguous sequence, including: i) an alignment score in relation to a reference genome, ii) an alignment score in relation to an assembly of alternate contiguous sequences, iii) a mapping quality of nucleotide reads, and iv) an amount of overlap with an SV genomic region. In addition, the call refinement system 106 can categorize reads based on their alignment according to the following categories: i) perfect alignment to an assembly of alternate contiguous sequences (e.g., satisfying a first alignment score threshold), ii) perfect alignment to a reference genome, iii) strong alignment to an assembly of alternate contiguous sequences (e.g., satisfies a second alignment score threshold but not the first alignment score threshold), iv) strong alignment to a reference genome (e.g., also satisfying the second alignment score threshold but not the first alignment score threshold), and v) no strong alignment either an assembly of alternate contiguous sequences or a reference genome (e.g., fails to satisfy the second alignment threshold in relation to both the assembly of alternate contiguous sequences and the reference genome). Based on these five categories, the call refinement system 106 can further determine fractions comparing each of these categories to determine a fraction of nucleotide reads (e.g., a fraction of reads overlapping with a target genomic region) supporting an alternate contiguous sequence versus a fraction of the nucleotide reads supporting a reference genome.

In addition, the call refinement system 106 determines, as a read-based sequencing metric, a number of split nucleotide reads from the nucleotide reads corresponding to the initial structural variant call. More particularly, the call refinement system 106 determines a number of nucleotide reads with no contiguous alignment (or less than a threshold number of bases that align) with a primary assembly of a reference genome, but that rather contain nucleotide-read fragments that align with two or more reference sequences within the reference genome. For example, the call refinement system 106 determines, using the call generation model 306, a split read count supporting a genotype call. For heterozygous deletion calls, a subset of false positive cases have large split read counts that exceed those in true positive cases, along with a coverage depth that is higher than expected. The call refinement system 106 can thus generate a split nucleotide read metric based on the nucleotide reads supporting a genotype call.

In some embodiments, the call refinement system 106 compares split read evidence supporting alternate alleles for forward and reverse oriented nucleotide reads, respectively. If most of the evidence is from either the forward or reverse oriented reads, this bias could be indicative of a systematic issue especially when the read count is relatively high (e.g., greater than 10 nucleotide reads). The call refinement system 106 uses forward and reverse read counts with perfect alignment scores with the contiguous sequence as sequencing metrics for the structural variant refinement machine-learning model.

Further, the call refinement system 106 can determine, as a read-based sequencing metric, a coverage depth of the nucleotide reads corresponding to the initial structural variant call. For example, the call refinement system 106 determines a count or a number of nucleotide reads that overlap with a target genomic region corresponding to a structural variant identified as present or absent by the initial structural variant call. Accordingly, coverage depth may be represented by a raw count of nucleotide reads overlapping with a target genomic region by at least a threshold number of nucleotide bases.

Further, the call refinement system 106 can determine, as part of the read-based sequencing metrics, an additional structural variant call located within a threshold number of base pairs from the initial structural variant call within the genomic sample. For example, the call refinement system 106 determines a structural variant call (e.g., a small size structural variant call), such as an insertion or a deletion within a threshold proximity (e.g., within 200 base pairs) of the initial structural variant call 308. Accordingly, the call refinement system 106 may indicate a presence or absence of such an additional structural variant call using a code, such as a binary code of 0 for absent and 1 for present.

In some embodiments, the call refinement system 106 further determines, as a read-based sequencing metric, an alignment of a contiguous sequence corresponding to the nucleotide reads with a reference sequence of a reference genome modified to include a structural variant corresponding to the initial structural variant call. In particular, the call refinement system 106 modifies the reference genome by changing nucleotide bases to reflect a structural variant, while excluding SNPs and indels in flanking regions. In theory, the modified reference genome may align perfectly with an alternate contiguous sequence, which provides some training benefit to a structural variant refinement machine-learning model in accurately identifying structural variants.

To modify a reference genome to include a structural variant, the call refinement system 106 can perform various steps. In particular, the call refinement system 106 can remove a portion of a sequence corresponding to the SV region (e.g., a deletion region for a deletion structural variant) from the reference genome. In some cases, the call refinement system 106 replaces the relevant portion of the reference sequence in a FAST-All (FASTA) file with a contiguous sequence representing the relevant structural variant. The call refinement system 106 can then regenerate the hash table using the modified FASTA file. In addition, the call refinement system 106 can run mapping-and-alignment components of a call generation model on the modified reference genome. The call refinement system 106 can further re-run variant caller components of the call generation model on the new mapping-and-alignment output.

For candidate structural variants where read-based evidence falls below a threshold (e.g., less than 5 or 10 nucleotide reads supporting a candidate structural variant call), one approach to finding missing reads is to modify the local reference sequence by replacing it with the contiguous sequence representing the candidate structure variant. For a true positive case, when reads are remapped with the modified reference genome, some of the nucleotide reads that were incorrectly mapped/aligned to the primary assembly of the reference genome would have a higher likelihood to be mapped correctly with a contiguous sequence representing the candidate structural variant and thereby increasing the read depth on the new modified reference genome. Based on the new mapping, if the call refinement system 106 reruns the call generation model, the call generation model 306 does not call a structural variant for a true homozygous deletion or an insertion for a true heterozygous deletion case. Additionally, the depth of read coverage should increase for the contiguous sequence representing the candidate structural variant relative to the original primary assembly, which should result in a more accurate variant call. The likelihood of achieving more accurate mapping could be estimated by aligning read length segments of the contiguous sequence representing the candidate structural variant to the reference genome.

In some embodiments, the call refinement system 106 analyzes flanking regions of a structural variant (as called by the call generation model) within a sample sequence, where the flanking regions include base calls within a threshold proximity (e.g., within 200 base pairs) of the structural variant. For example, the call refinement system 106 determines an initial structural variant call using a call generation model (e.g., a DRAGEN SV caller), modifies a reference genome to include a (portion of a) contiguous sequence that reflects the structural variant, and identifies flanking regions of a threshold size of 200 base pairs on either side of the structural variant. The call refinement system 106 further analyzes the flanking regions (e.g., the left flank and the right flank) of the combined sequence to determine the presence or absence of structural variants. Indeed, the call refinement system 106 can quantify the extent (e.g., the quantity, the magnitude, and/or the size) of single nucleotide polymorphisms (SNPs) and/or insertions or deletions (indels) based on a modified reference genome (e.g., the combined sequence of the reference genome and the contiguous sequence).

In some cases, the interpretation of a contiguous sequence is sensitive to scoring parameters and penalties within a Smith-Waterman algorithm. Accordingly, in these or other cases, the call refinement system 106 measures sensitivity to Smith-Waterman scoring parameters/penalties using deletion counts from Concise Idiosyncratic Gapped Alignment Report (CIGAR) string outputs of multiple scoring parameter sets. The call refinement system 106 can further use a maximum contiguous deletion length as well as the sum of all deletions corresponding to the genomic region spanned by the breakends as sequencing metrics (e.g., read-based sequencing metrics).

In some cases, the call refinement system 106 determines a read-based sequencing metric in the form of a deletion length in nucleotide bases based on one or more soft clipped nucleotide reads. For instance, the call refinement system 106 re-aligns soft clipped segments from nucleotide reads to determine a deletion length (or a length of a different type of structural variant). In some embodiments, the call refinement system 106 re-aligns only soft clipped portions of reads to provide an estimate of a length of a deletion or some other structural variant. For example, the call refinement system 106 performs re-alignment only if a size of a soft clipped portion satisfies (e.g., is greater than) a threshold number of soft clipped bases (e.g., 10 soft clipped bases or 20 soft clipped bases).

Additionally, in some embodiments, the call refinement system 106 determines or computes a re-alignment offset for soft clipped segments (e.g., those that satisfy the length requirement) by: i) for soft clipped reads to the left of a called structural variant, aligning the soft clipped portion to the left of a current position/coordinate denoting the end of the soft clipping, ii) for soft clipped reads to the right of a called structural variant, aligning the soft clipped portion to the right of a current position/coordinate denoting the start of the soft clipping, iii) determining a distance in number of nucleotide bases between an aligned position/coordinate and a location of soft clipping from an original mapping, iv) determining a left mode and a right mode for all distances determined via steps i)-iii), and v) determining a left re-alignment offset and a right re-alignment offset by determining a difference between the left mode and deletion length determined by the call generation model 306 (e.g., DRAGEN SV Caller) and between the right mode and the deletion length determined by the call generation model 306 (e.g., DRAGEN SV Caller), such as a number of nucleotide bases determined from variant length—alt seq length.

Further, the call refinement system 106 can determine a read-based sequencing metric in the form of a number of the nucleotide reads that exhibit a mapping quality metric that fails to satisfy a threshold mapping quality metric. To elaborate, the call refinement system 106 corrects for cases where a true positive shows nucleotide reads with low MAPQ scores (i.e., below a threshold MAPQ) that are nevertheless correctly mapped (although local alignment may be incorrect). In some cases, the call refinement system 106 utilizes MAPQ as a soft weighting to indicate likelihood of aligning with an alternate contiguous sequence or a reference genome. The call refinement system 106 can further determine a count or a number of reads with mapping quality metrics (e.g., MAPQ scores) that fail to satisfy (or are below) a threshold mapping quality metric (e.g., MAPQ=10 or MAPQ=60 or a relative MAPQ threshold). In some cases, the call refinement system 106 determines or generates a structural variant call based on the number of reads with low mapping quality metrics. In certain embodiments, such as in cases where MAPQ=60, the call refinement system 106 further incorporates an XQ score to determine an extended range on the likelihood of a structural variant. The call refinement system 106 can determine and incorporate a standard deviation of XQ across locally mapped reads for improved prediction of the structural variant refinement machine-learning model.

As further noted above, in some embodiments, the call refinement system 106 also determines an insert size representing a length of nucleotide-read fragments corresponding to an initial structural variant call determined by the call generation model 306. Specifically, the call refinement system 106 determines sizes or lengths (e.g., numbers of base pairs) for insertions (or other structural variants) within genomic region (e.g., an SV region) of a genomic sample.

In some cases, the call refinement system 106 determines a read-based sequencing metric in the form of a palindrome metric. For instance, the call refinement system 106 analyzes a portion of a reference sequence corresponding to a target genomic region where a structural variant is called (e.g., by a call generation model). Specifically, if the reference sequence in such a target genomic region is a palindrome (or within a threshold percentage of—or within a threshold number of base pairs from—a palindrome), then the likelihood of a folding effect increases. Based on the analysis, the call refinement system 106 identifies or detects fragments or portions of a genomic sample (e.g., sub-sequences of reads) within a threshold distance (e.g., within 200 base pairs) from one another and that are palindromes (which can exhibit a deletion due to a folding effect during base calling). The call refinement system 106 can determine or measure a distance or a closeness of (e.g., a number of base pairs separating) the segments of the palindrome metric. In some cases, the call refinement system 106 further incorporates a permutation entropy with the palindrome metric such that a palindrome match (e.g., a pair of segments exhibiting a palindrome of each other) with higher permutation entropy increases a likelihood of a deletion (or some other structural variant).

Further, in some embodiments, the call refinement system 106 determines a read-based sequencing metric in the form of a structural-variant likelihood representing a ratio of the initial structural variant call to a reference call for the one or more genomic coordinates based on an insert size. In particular, assuming there is no structural variant, then there is a certain implied insert size or fragment size. On the other hand, assuming there is a structural variant, then there is a different implied insert size or fragment size. Thus, based on a mean and a standard deviation of a fragment size, the call refinement system 106 can determine which is more likely between a presence or absence of a structural variant. For instance, in some embodiments, the call refinement system 106 determines a ratio of an initial structural variant call to a reference call for the one or more genomic coordinates according to the following formula:

$$\frac{{\prod }_{k = 0}^{{N}_{{A}^{- 1}}} {e}^{- \frac{{( {\tilde{l}}_{R , k} - {μ}_{I} )}^{2}}{2 {σ}_{I}^{2}}}}{{\prod }_{k = 0}^{{N}_{{A}^{- 1}}} {e}^{- \frac{{( {l}_{R , k} - {μ}_{I} )}^{2}}{2 {σ}_{I}^{2}}}}$$

where N_{A} is the number of reads showing evidence to support an alternate allele, l_{R,k} is the original estimated insert size corresponding to read k assuming no structural variant is present, l̅_{R,k} is the new estimated insert size based on alignment to the assembly of alternate contiguous sequences, μ_{I} is the mean insert size of a structural variant for the genomic sample, and σ_{I} is the standard deviation of the insert size of the structural variant for the genomic sample assuming a Gaussian distribution. In some cases, \tilde{l}_{R,k} is affected by the orientation of the split read and alignment relative to a candidate deletion (or another type of structural variant).

Depending on read orientation and alignment relative to a candidate SV genomic region, the call refinement system 106 may subtract length of a proposed structural variant (e.g., deletion) from an original insert size estimate (e.g., based on reference mapping and alignment). When considering all nucleotide reads providing alternate allele supporting evidence, the call refinement system 106 can determine the likelihood ratio (e.g., for alt vs. ref) based on projected insert sizes across the set of reads.

In some cases, the estimation of \tilde{l}_{R,k} is affected by the orientation of a split read serving as evidence for a structural variant (e.g., a deletion). Thus, the call refinement system 106 adjusts insert size estimates based on read orientation (e.g., for forward and reverse cases). However, the contiguous sequence often will not match reference flanking regions. Thus, the insert sizes computation will depend on both read orientation and the start location of the split read relative to breakend after aligning with the contiguous sequence. Additionally, the reference starts (e.g., genomic coordinate for start of a structural variant) provided in a BAM file often do not include the soft clipped portions of the nucleotide reads, and because the insert size computation uses the actual start of the reads, the call refinement system 106 adjusts reference starts to account for the amount of soft clipped bases.

In one or more embodiments, the call refinement system 106 determines a read-based sequencing metric in the form of a confidence interval around ending breakpoints. In particular, the call refinement system 106 utilizes the call generation model 306 to determine a confidence interval as a measure of certainty of a breakpoint location. For example, the call refinement system 106 determines a range of reference coordinates where a breakpoint might be located corresponding to a structural variant call. In some cases, the call refinement system 106 determines the range of reference coordinates to reflect a threshold percentile (e.g., the 95th percentile) in terms of confidence interval.

In certain embodiments, the call refinement system 106 further determines additional or alternative read-based sequencing metrics. For example, the call refinement system 106 determines a homology length as a read-based sequencing metric. Specifically, the call refinement system 106 determines a length of a nucleotide base sequence that is repeated in a target genomic region of a structural variant and/or a length of a nucleotide base sequence with at least a threshold measure of homology with other nucleotide base sequences (of similar lengths) within the target genomic region of the structural variant (e.g., HOMLEN=8 GCTTGAAC GCTTAAAC GCTAGAAC GCTTGAAC GCTTGTAC, etc.). In some cases, the call refinement system 106 determines a length of an inserted nucleotide base sequence as a read-based sequencing metric. In these or other cases, the call refinement system 106 determines a homology of an inserted nucleotide base sequence relative to a reference sequence within a target genomic region of a structural variant.

### B. Reference-Based Sequencing Metrics

As further illustrated in FIG. 3, in addition to read-based sequencing metrics, the call refinement system 106 can further determine or identify reference-based sequencing metrics 301 from a reference database 300. In particular, the call refinement system 106 determines the reference-based sequencing metrics 301 by analyzing one or more genomic regions of a reference genome corresponding to (or aligning with) the one or more genomic coordinates for the initial structural variant call 308.

Many challenging structural variant calls occur in low complexity genomic regions of the reference genome. In some cases, these genomic regions are characterized by some combination of multiple instances of long repeat sequences (e.g., more than 50 base pairs), very high number (e.g., more than 10) of shorter repeat sequences (e.g., 4-8 repeated bases), and on occasion containing a subset of the bases (e.g. As and Ts but no Cs or Gs). The nucleotide reads that are aligned correctly to such low complexity genomic regions often have portions or fragments of the nucleotide reads that map to a more unique sequence flanking a repeat-heavy region. Alternatively, a reference genome or genomic sample may include some intermediate breaks (e.g., single bases in between the primary repeat pattern that breaks the repetitiveness) that help with alignment of nucleotide reads with a low complexity genomic region of a reference genome. However, when combined with SNPs, indels, and sequencing errors, the alignment and the collection of reads with sufficient evidence to compare reference versus alternate allele support becomes problematic. Thus, in some embodiments, the call refinement system 106 monitors reference-based sequencing metrics (associated with complexity) which can be augmented with read-based sequencing metrics to provide an overall assessment of the likelihood of the presence of a structural variant (for both Bayesian and machine-learning approaches).

For example, the call refinement system 106 accesses or determines sequencing information about a particular reference genome (e.g., stored within the reference database 300 or the database 116). In some cases, the call refinement system 106 determines reference-based sequencing metrics including a tandem repeat length in nucleotide bases of a target genomic region within a reference genome corresponding to a candidate SV region of a genomic sample. Specifically, the call refinement system 106 analyzes portions of a reference genome that correspond to SV regions of a genomic sample to identify tandem repeats (e.g., sequences of two or bases that are repeated numerous times in a head-to-tail manner) and to further determine lengths (e.g., numbers of base pairs) within the tandem repeats.

In certain embodiments, the call refinement system 106 determines a reference-based sequencing metric in the form of a repetitiveness metric or homopolymer metric. Indeed, one indicator of a likelihood of a mis-mapping that needs to be corrected (e.g., a mis-mapping that results in a false positive) is based on repetitiveness of bases within a reference sequence. Thus, the call refinement system 106 can utilize various sequencing metrics to measure this repetitiveness, including: i) a maximum repeat pattern length that indicates the maximum length of a sequence of bases that is repeated at least two times over the span of the (reference genome corresponding to the) candidate SV region, ii) a maximum repeat length percentage that indicates the percentage of the (portion of the reference genome corresponding to the) SV region that is consumed or occupied by the maximum repeat pattern length, and iii) a maximum homopolymer length that indicates the length of the longest sequence of the same base in the (portion of the reference genome corresponding to the) candidate SV region.

In addition or in the alternative to a repetitiveness metric, in some cases, the call refinement system 106 determines a reference-based sequencing metric in the form of a permutation entropy of nucleotide bases. For example, the call refinement system 106 determines a measure of randomness of nucleotide sequences, which can be predictive of mapping/alignment accuracy. In some cases, the call refinement system 106 determines a permutation entropy by determining an entropy over permutations of a nucleotide sequence of a given length. For instance, the call refinement system 106 can determine permutation entropy according to the following formula:

S₁∈{A, C, G, T}

S₂∈{AA, AC, AG, AT, CA, CC, CG, CT, GA, GC, GG, GT, TA, TC, TG, TT}

S₃∈{AAA, AAC, AAG, AAT, ACT, . . . , TTA, TTC, TTG, TTT}

S₄∈{AAAA, AAAC, AAAG, AAAT, AACA, . . . , TTGT, TTTA, TTTC, TTTG, TTTT}

where S_{N} is a set of all permutations of length N base sequences, and where:

|S_{N}|=4^{N}

such that the probability of permutation element s_{N,k} occurring from set S_{N} is given by:

$${P}_{N , k} = \frac{{c}_{k}}{M - N + 1}$$

where cₖ is the number of occurrences of permutation element s_{N,k} in a sequence of length M. In some cases, the call refinement system 106 normalizes the permutation entropy as:

$${E}_{N} = \frac{- {Σ}_{k ∈ K} {p}_{N , k} {log}_{2} {p}_{N , k}}{2 N}$$

where K⊆{0, . . . , 4^{N}−1} is the set of indices such that p_{N,k}>0.

Beyond permutation entropy, the call refinement system 106 can further determine a reference-based sequencing metric in the form of identifying a presence or absence of a cytosine quadruplex (C-quadruplex) or a guanine quadruplex (G-quadruplex) in a target genomic region. To elaborate, the call refinement system 106 determines counts of cytosine calls and guanine calls within a target genomic region of a reference genome corresponding to an SV region of a genomic sample or genomic region under consideration for an initial structural variant call. To identify a cytosine quadruplex, the call refinement system 106 identifies occurrences (within the target genomic region) of four or more instantiations of three consecutive cytosine bases separated by one or more different nucleotide bases (e.g., a pattern of CCC A CCC A CCC A CCC). Similarly, to identify a guanine quadruplex, the call refinement system 106 identifies occurrences (within the target genomic region) of four or more instantiations of three consecutive guanine bases separated by one or more different nucleotide bases (e.g., a pattern of GGG T GGG T GGG T GGG). In one or more embodiments, the call refinement system 106 identifies a C-quadruplex or a G-quadruplex where up to a threshold number of nucleotide bases (e.g., up to 7 nucleotide bases) occur between instantiations of triple Cs or triple Gs. For instance, the call refinement system 106 identifies GGG TACC GGG TGTACA GGG AAGTCT GGG as a G-quadruplex. In some cases, G-quadruplexes (and C-quadruplexes) are known to cause issues with sequencing. Accordingly, the call refinement system 106 uses the presence of such sequences to adjust the confidence in the mapping and alignment of reads and the accuracy of subsequent contiguous sequence construction.

In certain embodiments, the call refinement system 106 determines a data compression metric as part of the reference-based sequencing metrics. In particular, the call refinement system 106 determines a data compression metric that quantifies a measure of randomness of a sequence using one or more data compression algorithms. One such data compression algorithm for lossless compression is the Liv-Zempel-Welch algorithm. Using this algorithm, the call refinement system 106 builds a dictionary of unique k-mers starting with length of one and comes up with an encoding for each entry in the dictionary. The call refinement system 106 can utilize the number of keys in the dictionary for the structural variant and the flanking regions in the reference genome as a sequencing metric.

In addition or in the alternative to the reference-based sequencing metrics noted above, in some embodiments, the call refinement system 106 determines a structural variant sequence alignment metric as part of the reference-based sequencing metrics. For instance, the call refinement system 106 uses gapless alignment scoring and Smith-Waterman alignment scoring of a proposed deletion sequence against the left/right flanking genomic regions in the reference. If there are multiple alignments that score above a threshold gapless alignment score and/or a threshold Smith-Waterman alignment score, the structural variant refinement machine-learning model may process a structural variant sequence alignment metrics as an indicator that there is a higher likelihood of an imprecise structural variant call.

Further, the call refinement system 106 can also determine a simulated read alignment metric as a reference-based sequencing metric. Assuming that the contiguous sequence representing or including a structural variant is accurate, there should theoretically be many nucleotide reads with good alignment to the contiguous sequence, even for heterozygous deletions. However, for low evidence true-positive cases of structural variants, there is a likelihood of missing reads because the reads corresponding to the SV region were either mapped elsewhere or unmapped. The call refinement system 106 can thus determine a likelihood of missing reads by simulating reads.

Specifically, the call refinement system 106 chooses segments from the contiguous sequence equal in length to the SBS reads. The call refinement system 106 chooses segments of the contiguous sequence that cross the breakend(s), that are equivalent to SBS read length, and that are aligned to the reference sequence in the SV region. For cases where alignment is ambiguous, alternate alignment scores will be higher and can serve as a possible guide for expected read depth. The call refinement system 106 can further use the segment of the contiguous sequence equivalent to read length that is symmetric about the breakend to obtain the highest alignment scores. The call refinement system 106 can further determine additional offsets from this symmetric point to check alternate alignment scores for a range of overlaps.

### C. Variant Region Quality Sequencing Metrics

As further illustrated in FIG. 3, the call refinement system 106 can determine variant region quality sequencing metrics as part of the sequencing metrics 304 or the sequencing metrics 310. More specifically, in some embodiments, the call refinement system 106 generates a subset of variant region quality sequencing metrics from sequencing data utilizing the call generation model 306. For example, the call refinement system 106 extracts or determines sequence data based on the read processing and mapping. In some cases, the call refinement system 106 generates sequence data as part of one or more digital files, such as BCL and FASTQ files, as described above in relation to the sequencing metrics 304.

In certain embodiments, the call refinement system 106 implements, utilizes, or applies the call generation model 306 to process or analyze sequence data. Indeed, in some embodiments, the call refinement system 106 generates a subset of variant region quality sequencing metrics by utilizing the call generation model 306 to re-engineer raw sequencing metrics (e.g., unmodified sequencing metrics within the sequence data). In particular, the call generation model 306 includes mapping-and-alignment components to map and align nucleotide base calls from the sequence data. In addition, the call generation model 306 includes variant calling components to generate the initial structural variant call 308 from the sequence data. In some cases, the call refinement system 106 extracts the variant region quality sequencing metrics that have been generated utilizing the mapping-and-alignment components and the variant calling components of the call generation model 306.

As an example of a variant region quality sequencing metric, the call refinement system 106 can determine a number of nucleotide reads that comprise at least a threshold number of base calls and correspond to a target genomic region for the initial structural variant call. For example, the call refinement system 106 analyzes sequence data to count base calls within nucleotide reads from the genomic sample (e.g., via the sequencing device 302 and/or the call generation model 306) corresponding to the initial structural variant call 308. The call refinement system 106 can further identify and count reads that include at least a threshold number of base calls. In some cases, the call refinement system 106 determines a read count threshold metric to quantify or indicate that the number of reads with at least a threshold number of base calls does not satisfy a read count threshold.

In addition or in the alternative to such a read count, in some embodiments, the call refinement system 106 determines, as a variant region quality sequencing metric, a base quality measure for reads with soft clipping in candidate SV regions. For example, the call refinement system 106 determines a soft clip read count as a number of soft clipped nucleotide reads within a candidate SV region (a.k.a target genomic region) of a genomic sample. In addition, the call refinement system 106 determines a low base call quality count as a number of calls with base call quality scores below a threshold base call quality score (e.g., a Q score or a QUAL score of 20, 30, 35, or 40) for a soft clipped portion of a nucleotide read. Further, the call refinement system 106 determines a count of low quality reads as a number of nucleotide reads with a low base call quality count that satisfies a threshold low base call quality count (e.g., a count of five base calls with a base call quality that is below a threshold base call quality score).

Further still, the call refinement system 106 determines a variant region quality sequencing metric in the form of a low quality read percentage that reflects a ratio of the low quality read count to the soft clip read count. In other words, the call refinement system 106 combines the low quality read count and soft clip read count described above in a ratio.

In addition or in the alternative to such read counts or ratios, in some embodiments, the call refinement system 106 determines, as a variant region quality sequencing metric, a number of nucleotide bases in an alternate contiguous sequence corresponding to a target genomic region from a reference genome for which based calls for nucleotide reads fail to satisfy a threshold base call quality score. Specifically, the call refinement system 106 can identify base calls that fail to satisfy a threshold base call quality score (e.g., a Q score or a QUAL score of 20, 30, 35, or 40). The call refinement system 106 can further determine an alternate base call quality metric to quantify or indicate a number of low quality base calls that are used to derive bases for an alternate contiguous sequence. To this end, the call refinement system 106 can align reads in a candidate SV region of a genomic sample to an alternate contiguous sequence. In addition, the call refinement system 106 can, for each position in the alternate contiguous sequence, record a base call quality score from alternate supporting reads. Further, the call refinement system 106 can, for each position in the alternate contiguous sequence, determine a median base call quality score from recorded base call quality scores for that position in the alternate supporting reads. The call refinement system 106 can further count the number of calls with base call quality scores that are below the threshold base call quality score (e.g., Q20, Q30, or Q40).

In addition or in the alternative to the various read-based sequencing metrics, variant region quality sequencing metrics, and/or reference-based sequencing metrics described above, the call refinement system 106 uses certain sequencing metrics that rely on percentages instead of the counts or numbers described above. As noted above certain sequencing metrics are based on numbers or counts associated with various reads or other features. In the alternative or in addition to such sequencing metrics, in certain embodiments, the call refinement system 106 determines variations of certain sequencing metrics based on percent values by normalizing the numbers/counts based on coverage in a target genomic region associated with an initial structural variant call. For example, some such sequencing metrics may include, but are not limited to, (i) a percentage of split nucleotide reads from the nucleotide reads corresponding to the initial structural variant call, (ii) a percentage of nucleotide reads that overlap with a target genomic region corresponding to a structural variant identified as present or absent by the initial structural variant call, (iii) a percentage of nucleotide reads exhibiting a mapping quality metric that fails to satisfy a threshold mapping quality metric, (iv) a percentage of nucleotide reads that comprise at least a threshold number of base calls and correspond to a target genomic region for the initial structural variant call, or (v) a percentage of nucleotide bases in an alternate contiguous sequence corresponding to the target genomic region from a reference genome for which base calls for the nucleotide reads fail to satisfy a threshold base call quality score.

Based on one or more of the reference-based sequencing metrics 301, the sequencing metrics 304, the sequencing metrics 310, or the initial structural variant call 308, as further illustrated in FIG. 3, the call refinement system 106 can utilize a structural variant refinement machine-learning model 312. More particularly, the call refinement system 106 can utilize the structural variant refinement machine-learning model 312 to process or analyze one or more of such sequencing metrics and the initial structural variant call 308 to generate a false positive likelihood 314. For example, the call refinement system 106 utilizes the structural variant refinement machine-learning model 312 to generate, based on reference-based sequencing metrics, read-based sequencing metrics, variant region quality sequencing metrics, and the initial structural variant call 308, the false positive likelihood 314 that reflects a likelihood or a probability that an initial structural variant call (e.g., an initial small size structural variant call) made by the call generation model 306 (e.g., the initial structural variant call 308) is a false positive.

In one or more embodiments, the false positive likelihood 314 indicates a high likelihood that an initial structural variant call 308 is a false positive, in which case the call refinement system 106 can correct the initial structural variant call. In certain cases, however, the false positive likelihood 314 indicates a low likelihood (e.g., below a threshold likelihood) that an initial structural variant call is a false positive. Accordingly, the call refinement system 106 can reinforce or confirm the initial structural variant call 308 made by the call generation model 306. Such confirmation can provide utility for clinicians by reinforcing that the initial structural variant call 308 is more likely to be correct (given that both models have come to the same conclusion) and therefore more actionable for treatment or other measures.

In certain cases, the call refinement system 106 can utilize the false positive likelihood 314 for purposes other than (or in addition to) determining a modified structural variant call. For example, the call refinement system 106 can utilize the false positive likelihood 314 as input for the call generation model 306 to perform further processing (e.g., to make additional variant calls, nucleotide base calls, and/or for producing other metrics). Indeed, the call refinement system 106 can recursively utilize the false positive likelihood 314 as input for a subsequent processing stage using the call generation model 306 to regenerate a structural variant call (or some other call).

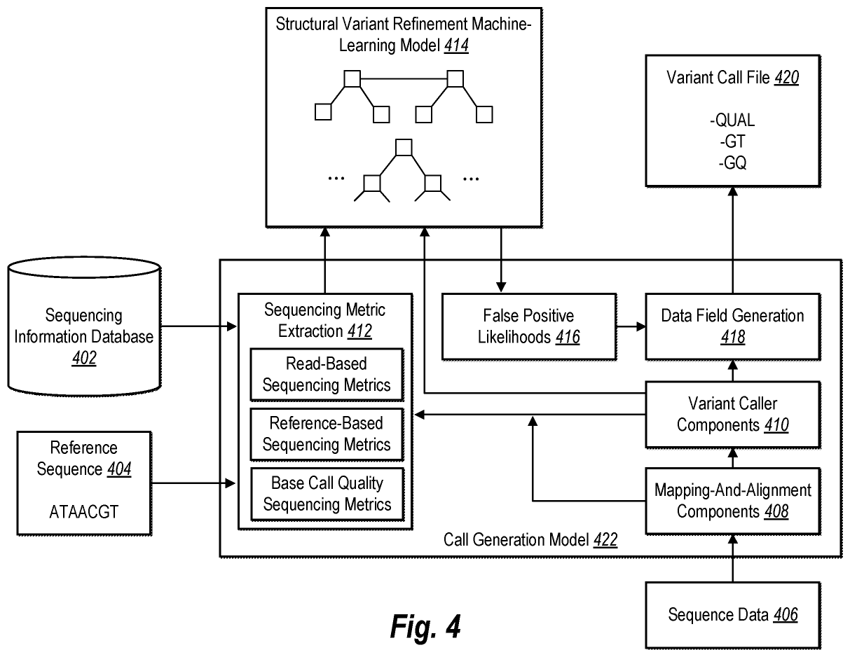

As mentioned, in certain embodiments, the call refinement system 106 utilizes a structural variant refinement machine-learning model together with a call generation model to generate a structural variant call (e.g., a small size structural variant call). In particular, the call refinement system 106 utilizes the structural variant refinement machine-learning model to modify data fields corresponding to a variant call file. FIG. 4 illustrates the call refinement system 106 generating a structural variant call by modifying a variant call file utilizing a structural variant refinement machine-learning model and call generation model in accordance with one or more embodiments.

In certain implementations, the call refinement system 106 determines, refines, or modifies an initial structural variant call based on the false positive likelihood 314. In some cases, the call refinement system 106 further considers additional or alternative factors to the false positive likelihood 314 in generating a modified structural variant call. For example, the call refinement system 106 utilizes metrics associated with single nucleotide variants (SNVs) and/or copy number variants (CNVs) to determine a modified structural variant call. Specifically, the call refinement system 106 determines SNV metrics, such as SNV calls within a threshold distance of an initial structural variant call, base-call quality scores associated with SNV calls, and other SNV metrics. In addition, the call refinement system 106 determines CNV metrics, such as CNV calls within a threshold distance of an initial structural variant call, base-call quality scores associated with CNV calls, and other CNV metrics. In some cases, the call refinement system 106 uses the SNV metrics and/or the CNV metrics (along with the false positive likelihood 314) to determine a refined or modified structural variant call. In certain embodiments, the call refinement system 106 can utilize SNV metrics and/or CNV metrics as further sequencing metrics to input into the structural variant refinement machine-learning model 312 for determining the false positive likelihood 314.

As illustrated in FIG. 4, the call refinement system 106 accesses a sequencing information database 402, a reference sequence 404 (e.g., a reference genome), and sequence data 406 extrapolated from one or more nucleotide reads. Indeed, the call refinement system 106 performs sequencing metric extraction 412 to extract or re-engineer sequencing metrics (e.g., read-based sequencing metrics, reference-based sequencing metrics, and variant region quality sequencing metrics) as described above in relation to FIG. 3. In some cases, the call refinement system 106 utilizes mapping-and-alignment components 408 of a call generation model 422 (e.g., the call generation model 306) to determine mapping-and-alignment metrics (e.g., as part of the read-based sequencing metrics, reference-based sequencing metrics, and/or variant region quality sequencing metrics). In addition, the call refinement system 106 utilizes variant caller components 410 of the call generation model 422 to generate variant calling metrics (e.g., as part of the read-based sequencing metrics, reference-based sequencing metrics, and variant region quality sequencing metrics). In some embodiments, the call refinement system 106 utilizes the variant caller components 410 of the call generation model 422 to likewise generate initial structural variant calls for one or more genomic coordinates of a genomic sample.

As further illustrated in FIG. 4, the call refinement system 106 generates false positive likelihoods 416. More specifically, the call refinement system 106 utilizes a structural variant refinement machine-learning model 414 to generate the false positive likelihoods 416 from the sequencing metrics and/or initial structural variant calls from the variant caller components 410. For example, the structural variant refinement machine-learning model 414 generates false positive likelihoods indicating likelihoods that initial structural variant calls of the call generation model 422 are false positives. As indicated above, in some embodiments, the call refinement system 106 determines a false positive likelihood by determining that an initial structural variant call is a false positive call or a true positive call based on the sequencing metrics.

From the false positive likelihoods 416, the call refinement system 106 further determines modified structural variant calls or confirms initial structural variant calls. Specifically, the call refinement system 106 determines a modified structural variant call by (i) changing an initial structural variant call from a positive structural variant call to a negative structural variant call based on the initial structural variant call being the false positive call or by (ii) changing an initial structural variant call from a negative structural variant call to a positive structural variant call based on the initial structural variant call being the true positive call.

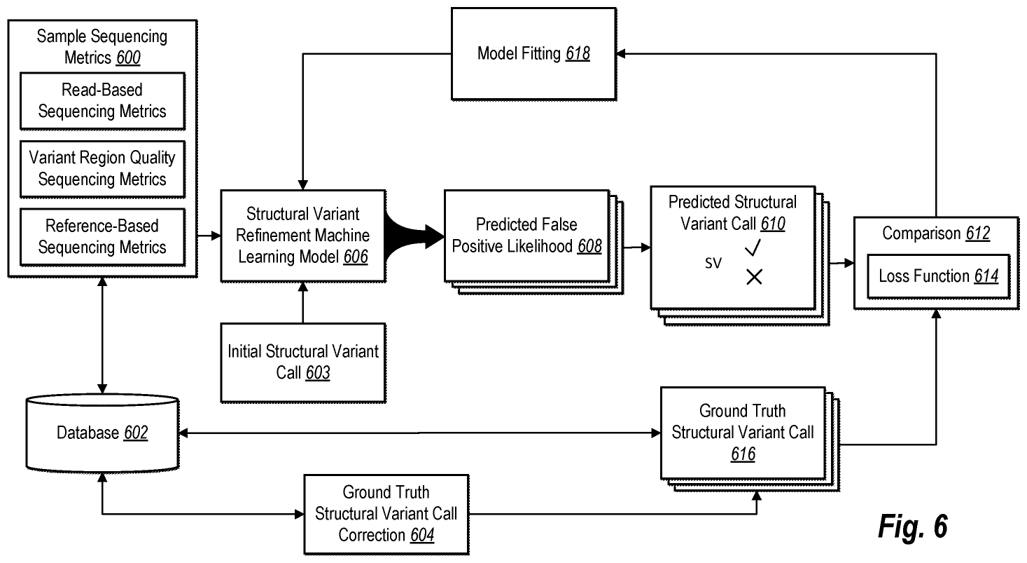

In some cases, the structural variant refinement machine-learning model 414 is an ensemble of gradient boosted trees that processes the sequencing metrics to generate the false positive likelihoods 416. For instance, the structural variant refinement machine-learning model 414 includes a series of weak learners, such as non-linear decision trees, that are trained in a logistic regression to generate the false positive likelihoods 416. In some cases, the structural variant refinement machine-learning model 414 includes metrics within various trees that define how the structural variant refinement machine-learning model 414 processes the sequencing metrics to generate the false positive likelihoods 416. Additional detail regarding the training of the structural variant refinement machine-learning model 414 is provided below with reference to FIG. 6.

In certain embodiments, the structural variant refinement machine-learning model 414 is a different type of machine-learning model, such as a neural network, a support vector machine, or a random forest. For example, in cases where the structural variant refinement machine-learning model 414 is a neural network, the structural variant refinement machine-learning model 414 includes one or more layers, some of which with neurons that make up the layer for processing the sequencing metrics. In some cases, the structural variant refinement machine-learning model 414 generates the false positive likelihoods 416 by extracting latent vectors from the sequencing metrics, passing the latent vectors from layer to layer (or neuron to neuron) to manipulate the vectors until utilizing an output layer (e.g., one or more fully connected layers) to generate the false positive likelihoods 416.

In addition (or in the alternative), the call refinement system 106 can determine the false positive likelihoods 416 by (i) utilizing an accumulation of statistical analyses over complex functions (depending on the architecture of the structural variant refinement machine-learning model 414) to determine how to best fit the data (e.g., based on relationship between the various sequencing metrics) or (ii) comparing other sequencing metrics, such as read depth, base-call quality scores, or others associated with a structural variant call with corresponding thresholds. For example, in some embodiments, the call refinement system 106 trains the structural variant refinement machine-learning model 414 to minimize a loss generated from a number of (different types of) sequencing metrics to determine weights and biases that best fit the data (e.g., that result in a reduced or minimized loss) for generating the false positive likelihoods 416.

As further illustrated in FIG. 4, the call refinement system 106 performs data field generation 418. More specifically, the call refinement system 106 generates data fields for a structural variant call utilizing the variant caller components 410 of the call generation model 422 and either modifies or maintains values for such data fields based the false positive likelihoods 416. For instance, the call refinement system 106 modifies various metrics, such as quality metrics, mapping metrics, or other metrics, associated with the structural variant call. In certain embodiments, the structural variant call is represented or defined by the variant call file 420 which includes metrics corresponding to the data fields, such as a call-quality metric corresponding to a call-quality field, a genotype metric corresponding to a genotype field, and a genotype-quality metric corresponding to a genotype-quality field. Other fields include a CIGAR string field, a read depth field, an ancestral allele field, and/or other variant call format fields.

In addition to generating an initial structural variant call via the call generation model 422, the call refinement system 106 also recalibrates or modifies the initial structural variant call based on the false positive likelihoods 416 from the structural variant refinement machine-learning model 414. In one or more implementations, the call refinement system 106 modifies the initial structural variant call by modifying or recalibrating data fields for one or more of the metrics associated with the nucleotide base call (e.g., as included within the variant call file 420).

As described, the call refinement system 106 generates false positive likelihoods 416 and a structural variant call from the same set of sequencing metrics (or a subset of the sequencing metrics that are shared between the structural variant refinement machine-learning model 414 and the call generation model 422) and/or an initial structural variant call from the variant caller components 410. Indeed, the call refinement system 106 utilizes the structural variant refinement machine-learning model 414 to generate the false positive likelihoods 416 from sequencing metrics while also generating an initial structural variant call for a genomic sample. Indeed, the call refinement system 106 can operate the structural variant refinement machine-learning model 414 in parallel with the call generation model 422 to generate metrics for an initial structural variant call and false positive likelihoods 416 for recalibrating the generated metrics.

In one or more implementations, the call refinement system 106 updates or otherwise modifies the data fields for the variant call file 420 according to particular algorithms. After modifying such data fields, the call refinement system 106 can generate the variant call file 420 (e.g., a post-filter variant call file) to include metrics reflecting the updated data fields for QUAL, GT, and GQ (or other VCF fields). For instance, in some cases, the call refinement system 106 updates the QUAL field for one or more structural variant calls based on the false positive likelihoods 416. As indicated above, in some cases, QUAL indicates the probability that there is some kind of variant (or other nucleotide base call) at a given location, measured in PHRED scale.

The call refinement system 106 can remove false positive structural variant calls and recover false negative structural variant calls by changing corresponding VCF metrics based on the false positive likelihoods 416. To remove a false positive structural variant call, in some cases, the call refinement system 106 decreases the quality metric (e.g., QUAL score) of a structural variant call that initially passed a quality filter—based on the false positive likelihoods 416 from the structural variant refinement machine-learning model 414. Based on determining the decreased quality metric falls below a threshold metric, the call refinement system 106 determines that the structural variant call no longer passes the quality filter. The call refinement system 106 thus filters out, or removes, the false structural variant call that initially passed the filter by changing a quality metric (or one or more other metrics).

To recover a false negative structural variant call, the call refinement system 106 increases the quality metric of a structural variant call that initially failed a quality filter—based on the false positive likelihoods 416 from the structural variant refinement machine-learning model 414. Based on determining the increased quality metric exceeds a threshold metric, the call refinement system 106 determines that the structural variant call passes the quality filter. The call refinement system 106 thus recovers a false negative structural variant call that was initially filtered out by changing its quality metric.

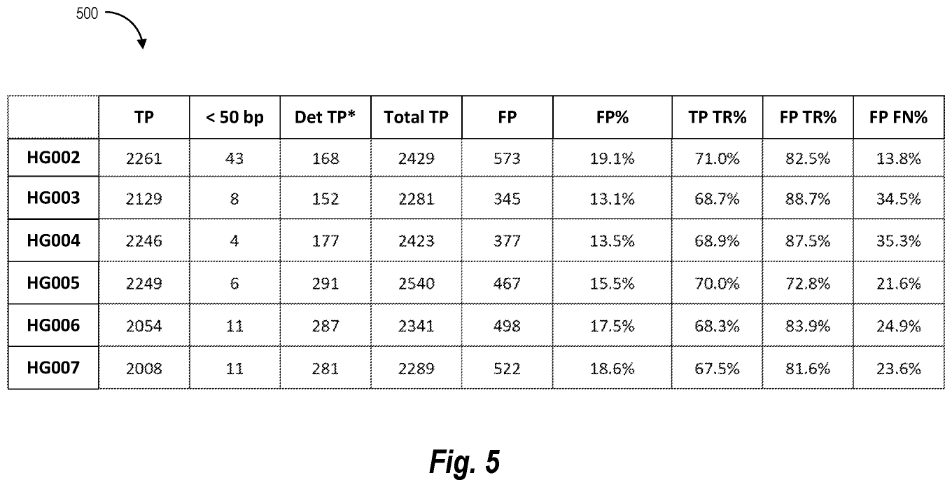

As just mentioned, the call refinement system 106 can improve accuracy in structural variant calling compared to prior systems. In particular, by using a structural variant refinement machine-learning model trained on the sequencing metrics described herein, the call refinement system 106 reduces or removes false positive structural variant calls and/or false negative structural variant calls by correcting structural variant calls initially made by a call generation model. FIG. 5 illustrates an example table of correcting structural variant calls using the structural variant refinement machine-learning model in accordance with one or more embodiments.

As illustrated in FIG. 5, researchers have demonstrated certain improvements of the call refinement system 106. To elaborate on experimental results, a table 500 includes rows corresponding to different datasets, such as HG002, HG003, HG004, HG005, HG006, and HG007, which are specific sets of available human genome data corresponding to genetics of various genomic samples. As shown, the table 500 includes a “TP” column indicating the number of true positive structural variant calls determined using a call generation model (e.g., the call generation model 422). The table 500 further includes a “Det TP” column that indicates a number of true positives detected (or recovered from false positives and/or false negatives) using a structural variant refinement machine-learning model (e.g., the structural variant refinement machine-learning model 414). Totaling the “Det TP” column and the “TP” column yields the “Total TP” column, where the “Total TP” column indicates a total number of true positive structural variant calls, including those determined via the call generation model and those recovered or refined via the structural variant refinement machine-learning model.

In addition, the table 500 includes a “<50 bp” column, which indicates a number of (false positive) structural variant calls that the call refinement system 106 filters out for fails to satisfy a minimum length threshold of at least 50 base pairs. In addition, the table 500 includes an “FP” column, which indicates a number of false positives that remain after the call refinement system 106 applies the call generation model and the structural variant refinement machine-learning model. Accordingly, totaling the “<50 bp” column, the “Det TP” column, and the “FP” column yields the total number of false positive structural variant calls before applying the structural variant refinement machine-learning model. Thus, as shown by the table 500, the call refinement system 106 reduces the number of false positive structural variant calls and increases the number of true positive structural variant calls for better accuracy in structural variant calling.

As mentioned above, in certain described embodiments, the call refinement system 106 trains a structural variant refinement machine-learning model to generate false positive likelihoods for correcting or confirming structural variant calls. In particular, the call refinement system 106 trains the structural variant refinement machine-learning model using specific training data that is tailored and engineered for the structural variant refinement machine-learning model. FIG. 6 illustrates an example diagram depicting a training process for the structural variant refinement machine-learning model in accordance with one or more embodiments.

As illustrated in FIG. 6, the call refinement system 106 determines or performs a ground truth structural variant call correction 604. To elaborate, the call refinement system 106 identifies, from a truth dataset (e.g., a dataset of reads and variant calls from a CCS Read-Based SV Caller), a ground truth structural variant call corresponding to a structural variant call that is incorrectly labeled as a false positive instead of a true positive. The call refinement system 106 identifies such a mislabeled ground truth structural variant call based on one or more truth set nucleotide reads for the ground truth structural variant call satisfying one or more structural variant criteria. The truth set nucleotide reads may include long nucleotide reads (e.g., CCS long reads or nanopore long reads) and/or short nucleotide reads. In some cases, the truth set nucleotide reads underlying the ground truth structural variant call include flanking regions upstream or downstream from a structural variant and/or is location-adjusted according to long reads in a truth dataset (e.g., from the database 602) to correct for ambiguities in potential sequence locations for structural variants. In certain embodiments, the call refinement system 106 performs a correction process to correct for ambiguities by identifying or detecting concordance between nucleotide reads used to generate a truth dataset and a contiguous sequence that corresponds to nucleotide reads (e.g., for a target genomic region) and that is generated by a call generation model—but that represents an alternate nucleotide base sequence. As suggested above, for instance, the call generation model (e.g., DRAGEN SV Caller) may generate a contiguous sequence corresponding to nucleotide reads with a reference sequence of a reference genome modified to include a structural variant corresponding to the initial structural variant call 603.

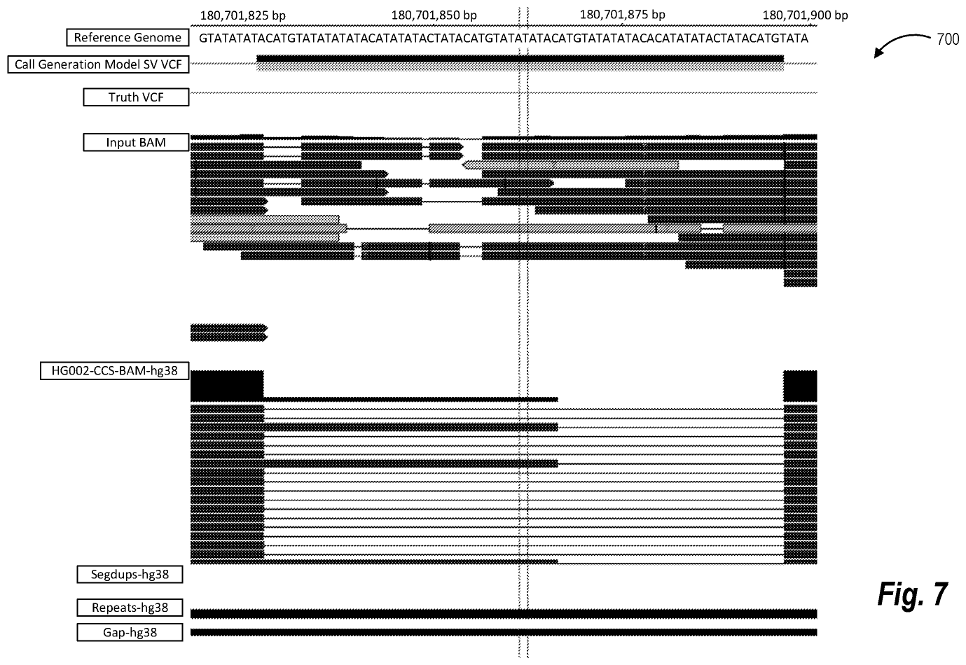

After identifying a mislabeled ground truth structural variant call, the call refinement system 106 further changes a label for the mislabeled ground truth structural variant call from false positive structural variant call to true positive structural variant call and uses the modified truth dataset (including the changed label) as training data for the structural variant refinement machine-learning model 606. Additional detail regarding determining the structural variant criteria and correcting ground truth data for training the structural variant refinement machine-learning model 606 is provided below in relation to FIG. 7.

As further illustrated in FIG. 6, the call refinement system 106 accesses sample sequencing metrics 600 and corrected ground truth structural variant calls (and/or other corrected training data) from a database 602 (e.g., the database 116). Accordingly, in some cases, the sample sequencing metrics 600 have a corresponding and corrected ground truth structural variant call 616 associated with them, where the ground truth structural variant call 616 indicates an actual structural variant call and its various metrics that result from the sample sequencing metrics. For instance, the call refinement system 106 utilizes the sample sequencing metrics 600 and the ground truth structural variant calls (e.g., the ground truth structural variant call 616) from a training dataset generated using the CCS Read-Based SV Caller. In the alternative, the training dataset includes metrics and structural variant calls from the U.S. Food and Drug Administration (FDA), called the PrecisionFDA dataset. In some cases, the sample sequencing metrics 600 include a subset of sample sequencing metrics for each structural variant call in a ground truth variant call file. The ground truth variant call file can have a ground truth variant call (e.g., genotype metric in a genotype field) and/or a ground truth structural variant call corresponding to each subset of sample sequencing metrics.

As further illustrated in FIG. 6, the call refinement system 106 generates a predicted false positive likelihood 608 based on the sample sequencing metrics 600 and further based on an initial structural variant call 603 (e.g., a structural variant call made by a call generation model). Specifically, the call refinement system 106 inputs the sample sequencing metrics 600 and the initial structural variant call 603 into the structural variant refinement machine-learning model 606 and utilizes the structural variant refinement machine-learning model 606 to generate the predicted false positive likelihood 608 from the sample sequencing metrics 600.

Based on the predicted false positive likelihood 608, the call refinement system 106 determines a predicted structural variant call 610. In some training iterations, the predicted structural variant call 610 either differs from or matches an initial structural variant call determined by a call generation model. As indicated above, the call refinement system 106 can utilize (i) a call generation model to generate an initial structural variant call and (ii) the structural variant refinement machine-learning model 606 to modify (data fields corresponding to a variant call file for) the structural variant call. Such modified or recalibrated values are output in a modified variant call file (VCF) by, for example, the call generation model.

As further illustrated in FIG. 6, the call refinement system 106 performs a comparison 612. Specifically, the call refinement system 106 performs the comparison 612 between (i) the predicted structural variant call 610 and (ii) the ground truth structural variant call 616. In some embodiments, the call refinement system 106 utilizes a loss function 614 to compare such structural variant calls (e.g., to determine an error or a measure of loss between them). For instance, in cases where the structural variant refinement machine-learning model 606 is an ensemble of gradient boosted trees, the call refinement system 106 utilizes a mean squared error loss function (e.g., for regression) and/or a logarithmic loss function (e.g., for classification) as the loss function 614.

By contrast, in embodiments where the structural variant refinement machine-learning model 606 is a neural network, the call refinement system 106 can utilize a cross entropy loss function, an L1 loss function, or a mean squared error loss function as the loss function 614. For example, the call refinement system 106 utilizes the loss function 614 to determine a difference between the predicted structural variant call 610 and the ground truth structural variant call 616.

As further illustrated in FIG. 6, the call refinement system 106 performs model fitting 618. In particular, the call refinement system 106 fits the structural variant refinement machine-learning model 606 based on the comparison 612. For instance, the call refinement system 106 performs modifications or adjustments to various parameters of the structural variant refinement machine-learning model 606 to reduce the measure of loss from the loss function 614 for a subsequent training iteration.

For gradient boosted trees, for example, the call refinement system 106 trains the structural variant refinement machine-learning model 606 on the gradients of the errors determined by the loss function 614. For instance, the call refinement system 106 solves a convex optimization problem (e.g., of infinite dimensions) while regularizing the objective to avoid overfitting. In certain implementations, the call refinement system 106 scales the gradients to emphasize corrections to under-represented classes (e.g., where there are significantly more true positives than false positive variant calls).

In some embodiments, the call refinement system 106 adds a new weak learner (e.g., a new boosted tree) to the structural variant refinement machine-learning model 606 for each successive training iteration as part of solving an optimization problem. For example, the call refinement system 106 finds a feature (e.g., a sequencing metric) that minimizes a loss from the loss function 614 and either adds the feature to the current iteration's tree or starts to build a new tree with the feature.

In addition or in the alternative to gradient boosted decision trees, the call refinement system 106 trains a logistic regression to learn parameters for generating one or more variant call classifications such as a true-positive classification. To avoid overfitting, the call refinement system 106 further regularizes based on hyperparameters, such as the learning rate, stochastic gradient boosting, the number of trees, the tree-depth(s), complexity penalization, and L1/L2 regularization.

In embodiments where the structural variant refinement machine-learning model 606 is a neural network, the call refinement system 106 performs the model fitting 618 by modifying internal parameters (e.g., weights) of the structural variant refinement machine-learning model 606 to reduce the measure of loss for the loss function 614. Indeed, the call refinement system 106 modifies how the structural variant refinement machine-learning model 606 analyzes and passes data between layers and neurons by modifying the internal network parameters. Thus, over multiple iterations, the call refinement system 106 improves the accuracy of the structural variant refinement machine-learning model 606.

In some embodiments, the call refinement system 106 adjusts weights of the structural variant refinement machine-learning model 606 based on structural-variant-call class imbalance to improve training. More specifically, the call refinement system 106 detects a structural-variant-class imbalance, such as at least a threshold difference (e.g., greater than a 20%, 45%, 55% difference in class) between the number of false positive structural variant calls and the number of true positive structural variant calls (e.g., the number of false positives is significantly less than the number of true positives). Based on detecting a structural-variant-class imbalance, the call refinement system 106 weights the gradient of the less frequent class (e.g., true positive structural variant calls) more heavily relative to the gradient of the more frequent class (e.g., false positive structural variant calls) during training. For example, the call refinement system 106 determines a scaling factor for weighting the gradients based on the ratio of false positive structural variant calls to true positive structural variant calls in a training dataset. In some cases, the call refinement system 106 dynamically adjusts the scaling factor based on changes to the ratio of false positive structural variant calls to true positive structural variant calls that may occur in a training dataset (e.g., a new training dataset).

By determining and applying a scaling factor for the structural variant refinement machine-learning model 606, the call refinement system 106 can dynamically adjust a sensitivity or true positive rate at which the call refinement system 106 determines structural variant calls based on false positive likelihoods from the structural variant refinement machine-learning model 606. Similarly, by determining and applying a scaling factor for the structural variant refinement machine-learning model 606, the call refinement system 106 can dynamically adjust F-1 score at which the call refinement system 106 classifies or determines structural variant calls (e.g., initial structural variant calls) based on false positive likelihoods from the structural variant refinement machine-learning model 606. Such a scaling factor can, for example, adjusts weights of the structural variant refinement machine-learning model 606 that make it more or less likely that a false positive likelihood (or a likelihood score) indicates an initial structural variant call is indeed a false positive or indeed indicates a particular structural variant is present at one or more genomic coordinates of a genomic sample.

Indeed, in some cases, the call refinement system 106 repeats the training process illustrated in FIG. 6 for multiple iterations. For example, the call refinement system 106 repeats the iterative training by selecting a new set of corrected training data along with a corresponding ground truth structural variant call. The call refinement system 106 further generates a new predicted false positive likelihood for each iteration along with a new predicted structural variant call. As described above, the call refinement system 106 also performs a comparison at each iteration and further performs model fitting. The call refinement system 106 repeats this process until the structural variant refinement machine-learning model 606 generates a false positive likelihood which results in a predicted structural variant call that satisfies a threshold measure of loss.

As mentioned above, in certain described embodiments, the call refinement system 106 generates a modified set of training data for adjusting parameters of a structural variant refinement machine-learning model. In particular, the call refinement system 106 modifies training data by correcting errors within a truth dataset, such as a dataset generated by the CCS Read-Based SV Caller and/or the PrecisionFDA dataset. FIG. 7 illustrates an integrative genomics viewer (IGV) chart for an example scenario where the call refinement system 106 corrects errors exhibited by a truth dataset in accordance with one or more embodiments.

As illustrated in FIG. 7, the IGV chart 700 depicts a target genomic region of a reference genome along input BAM file data (represented by the “Input BAM” area), circular consensus sequencing (CCS) nucleotide reads (represented by the “HG002-CCS-BAM-hg38” area), an indication of a Call Generation Model SV Caller call (represented by the “Call Generation Model SV VCF” area), and an indication of a structural variant call within a truth dataset (represented by the “Truth VCF” area). As shown, the truth dataset indicates that no structural variant exists for a genomic sample when compared to the depicted target genomic region of the reference genome. However, the Call Generation Model SV Caller has made a structural variant call for the same target genomic region. Further, other sequencing data (e.g., sequencing metrics) depicted in the IGV chart 700 indicate that a structural variant does indeed exist in the target genomic region shown. Relying on the truth dataset that reflects this incorrect call when training the structural variant refinement machine-learning model would be inaccurate and mis-train the structural variant refinement machine-learning model.

Accordingly, in some embodiments, the call refinement system 106 automatically (e.g., without user interaction for prompting or guiding) corrects the incorrect structural variant call to generate more reliable training data (e.g., more accurate ground truth structural variant calls). To correct the missed call in the truth dataset, the call refinement system 106 can determine that a ground truth structural variant call is incorrectly labeled as a false positive instead of a true positive. Indeed, the call refinement system 106 can determine that the ground truth structural variant call is incorrectly labeled by determining structural variant criteria associated with the ground truth structural variant call. Specifically, the call refinement system 106 analyzes sequencing data (e.g., the nucleotide reads and other information depicted in the IGV chart 700) to determine that the target genomic region of a genomic sample analyzed by a ground truth SV caller (e.g., the CCS Read-Based SV Caller) exhibits a structural variant where no such call was made.

In some cases, to make a correction, the call refinement system 106 determines that the nucleotide reads for the incorrect ground truth structural variant call satisfy one or more structural variant criteria. For example, the call refinement system 106 parses a Concise Idiosyncratic Gapped Alignment Report (CIGAR) string (e.g., a CIGAR string generated for the genomic sample and/or the reference genome) to identify a truth set nucleotide read (e.g., a CCS long read or nanopore long read) of the truth dataset that satisfies a threshold mapping quality metric. In addition, the call refinement system 106 determines a portion of the CIGAR string that includes or indicates a starting index of a structural variant call generated by a call generation model (e.g., the DRAGEN SV Caller) at a location where a call is missing in the truth dataset. Further, the call refinement system 106 determines that the starting index corresponds to a structural variant and matches a length (e.g., a number of base pairs) of the corresponding structural variant call generated by the call generation model (as shown in the IGV chart 700).

In one or more embodiments, as part of making a correction to a truth dataset, the call refinement system 106 compares flank length of truth set nucleotide reads on both sides of a structural variant call with a threshold flank length (e.g., a threshold number of base pairs). When searching for potential false positives in a truth dataset, the call refinement system 106 searches for truth set nucleotide reads (e.g., CCS long reads) whose alignment to a reference genome supports an initial structural variant call from a call generation model. For instance, the call refinement system 106 determines whether the following criteria are satisfied: i) the mapping quality metric for the truth set nucleotide read satisfies a threshold mapping quality metric and ii) the two ends of the truth set nucleotide read align outside a particular reference range of genomic coordinates. Specifically, the call refinement system 106 determines the reference range of genomic coordinates based on genomic coordinates of an initial structural variant call.

For instance, the call refinement system 106 determines a reference range of genomic coordinates defined by A−D to B+D, where A and B represent the ends of a structural variant call in reference genomic coordinates, and where D represents a minimum flank size threshold (e.g., 1,000 to 2,000 base pairs). The motivation to have a minimum flank size threshold is to increase the likelihood of the correct alignment of the truth set nucleotide read at the location of the structural variant. When the flank size is too short, a CCS long read or nanopore long read as the truth set nucleotide read is susceptible to alternative (and possibly inaccurate) alignments similar to short reads.

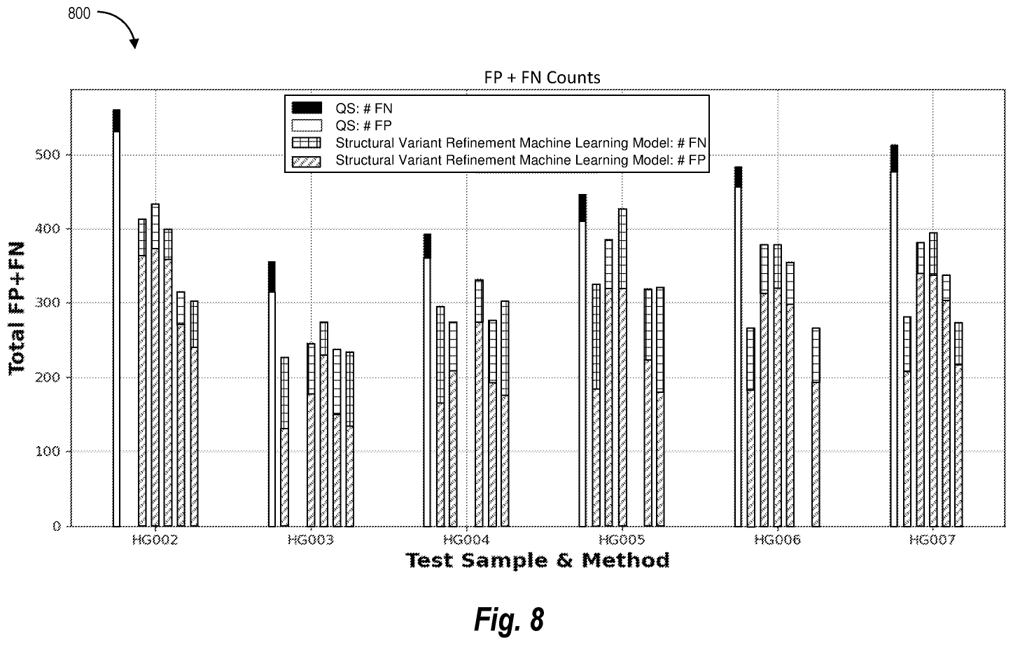

As mentioned above, in certain described embodiments, the call refinement system 106 trains a structural variant refinement machine-learning model using one or more training datasets. In particular, the call refinement system 106 utilizes a five-way split of training data for cross validation. FIG. 8 illustrates an example table depicting the split of training data for cross validation and corresponding performance of the structural variant refinement machine-learning model in accordance with one more embodiments.

As illustrated in FIG. 8, the table 800 shows six training datasets for genomic samples, HG002-HG007. The table 800 also depicts numbers of false positives and false negatives produced by the structural variant refinement machine-learning model when trained over the respective training datasets. The call refinement system 106 performs cross validation training by selecting one portion of each training dataset (e.g., ⅕ or 20%) to use as test data, while using the remaining portions (e.g., ⅘ or 80%) as training data for learning or adjusting model parameters. Indeed, the table 800 depicts gaps where the corresponding data portion is withheld for testing, that is, where the gap moves one place to the right for each training dataset to represent a different withheld portion for cross validation.

Because ground truth data for structural variants can be challenging to find in high volume and inaccurate when relying on a CCS Read-Based SV Caller as a proxy for ground truth, researchers used base-call quality scores (“QS”) for structural variant calls determined by a call generation model (e.g., DRAGEN SV Caller) as an approximate ground truth. In particular, as a point of comparison, the table 800 includes an estimate of false negative structural variant calls (“FN”) and false positive structural variant calls (“FP”) for the call generation model based on a threshold base-call quality score (“QS”), such as Q score 20 or Q score 30. As shown in table 800, positive structural variant calls with a base-call quality score below the threshold base-call quality score are counted as false positive structural variant calls. By contrast, negative structural variant calls with a base-call quality score below the threshold base-call quality score are counted as false negative structural variant calls. Table 800 counts false positive structural variant calls and false negative structural variant calls using the same approach for the call generation model both with and without modified structural variant calls using a structural variant refinement machine-learning model.

As shown, for each of HG002-HG007, the call refinement system 106 reduces the number of false negative structural variant calls and false positive structural variant calls by modifying structural variant calls based on false positive likelihoods output by a structural variant refinement machine-learning model compared to no such modified structural variant calls determined by the call generation model. In this example, the structural variant reference machine-learning model takes the form of XGBoost. For most genomic samples HG002-HG007, the call refinement system 106 shows a 25-50% reduction in FP+FN by using a structural variant refinement machine-learning model.

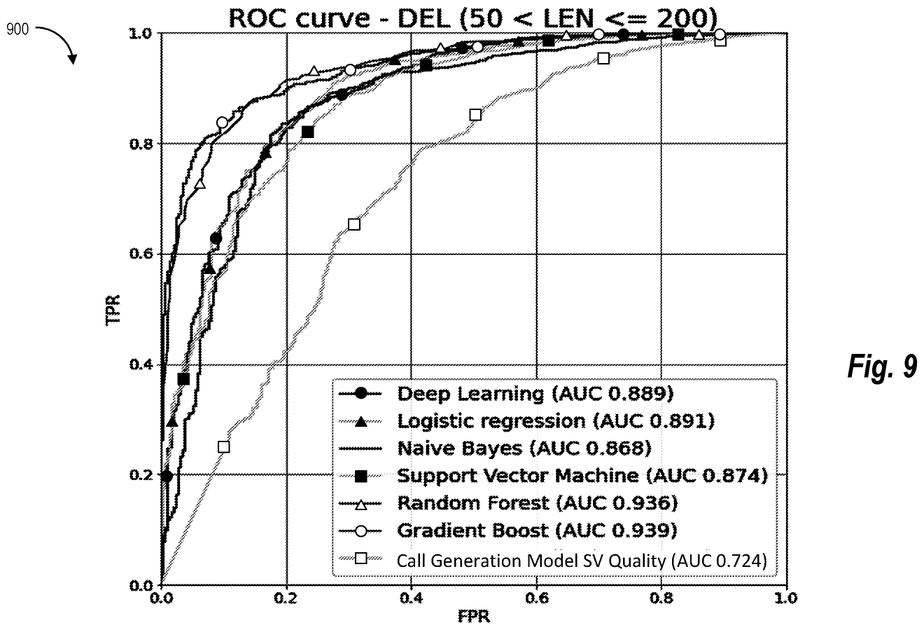

As just mentioned, researchers have demonstrated accuracy improvements of the call refinement system 106 in relation to prior systems. In particular, researchers have compared results when training various machine learning architectures using corrected truth datasets and sequencing metrics described herein. FIG. 9 illustrates an example graph of experimental results for various machine learning architectures for a structural variant refinement machine-learning model as compared to Call Generation Model SV Caller quality in accordance with or more embodiments.

As illustrated in FIG. 9, the receiver operating characteristic (ROC) curves in the graph 900 depict performance for various versions or architectures of the structural variant refinement machine-learning model. Specifically, the graph 900 depicts results from training different machine learning architectures to determine small size deletion calls for variants between 50 and 200 base pairs in length. For comparison, the graph 900 also illustrates performance of the Call Generation Model SV Caller. When evaluating the ROC curves, those that fit to the upper left of the graph 900 exhibit better performance, with higher true positive rates (“TPR”) and lower false positive rates (“FPR”).

As shown in FIG. 9, each version of the structural variant refinement machine-learning model outperforms the Call Generation Model SV Caller alone (e.g., the call generation model). In the illustrated experiment, the best performing architectures for the structural variant refinement machine-learning model are gradient boosted trees (e.g., XGBoost) and a random forest model, exhibiting the highest area under the curve (“AUC”).

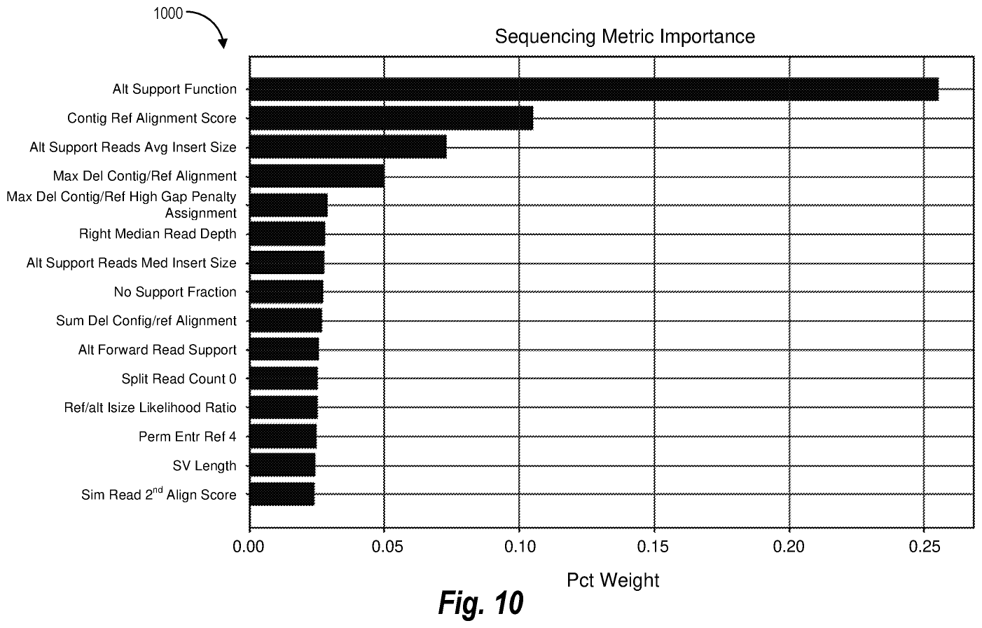

In certain described embodiments, the call refinement system 106 generates or determines importance measures associated with individual sequencing metrics. For example, an importance measure can refer to a measure of effect, influence, or impact that a sequencing metric has on a determination or prediction of a structural variant call. For example, an importance measure indicates how much of a role one sequencing metric plays in determining a nucleotide base call over a different nucleotide base call (and compared to other sequencing metrics). FIG. 10 illustrates an example graph depicting the importance measure of some sequencing metrics in accordance with one or more embodiments.

As illustrated in FIG. 10, the graph 1000 depicts a ranked order of sequencing metrics based on their respective importance measures (e.g., relative to deletions). For example, the call refinement system 106 determines an importance measure for each sequencing metric used to generate a deletion. In some cases, the call refinement system 106 determines different importance measures for the same sequencing metrics relative to a different type of structural variant. To determine importance measures, the call refinement system 106 determines a weight to apply to each sequencing metric given its impact on a resultant structural variant call determined via a structural variant refinement machine-learning model.

As shown, the graph 1000 depicts the “Alt Support Function” (e.g., a fraction of nucleotide reads with sufficient overlap of structural variant breakends that have perfect or near perfect alignment with an alternate contiguous sequence) as the most important sequencing metric with the highest weight. The graph 1000 further depicts importance measures for other sequencing metrics in descending order of importance for (determining deletions using) the structural variant refinement machine-learning model.

For a more complete list of sequencing metrics, including indications of their respective importance measures relative to different structural variants, the call refinement system 106 determines one or more of the following read-based sequencing metrics: i) an alt support fraction (high importance for deletions, high importance for insertions) which indicates a fraction of nucleotide reads with sufficient overlap of structural variant breakends that have perfect or near perfect alignment with an alternate contiguous sequence, ii) a left soft clip count (high importance for deletions, low importance for insertions) which indicates a count of nucleotide reads supporting an alternate sequence with the most common deletion length inferred from remapping the right soft clipped reads, iii) a nearby structural variant call (high importance for deletions, high importance for insertions) which indicates whether there is another structural variant call within a threshold number of base pairs of an initial structural variant call, iv) a low MAPQ count (high importance for deletions, high importance for insertions) which indicates a number of reads with perfect alignment with an alternate contiguous sequence that have at least a threshold mapping quality metric, v) insert size statistics (high importance for deletions, medium importance for insertions) which indicate mean and median insert sizes for nucleotide reads supporting an alternate sequence more than a reference sequence, vi) a soft right offset (high importance for deletions, medium importance for insertions) which indicates an offset between an estimation deletion length based on realignment of right soft clipped reads and Call Generation Model SV length (e.g., SV length as determined by a call generation model, such as DRAGEN SV Caller), vii) a right flank soft clip count (medium importance for deletions, high importance for insertions) which indicates a count of nucleotide reads supporting an alternate sequence with the most common deletion length inferred from remapping the right soft clipped reads, viii) a soft left offset (medium importance for deletions, low importance for insertions) which indicates an offset between an estimation deletion length based on realignment of left soft clipped reads and Call Generation Model SV length, ix) a quality score (medium importance for deletions, high importance for insertions) which indicates a quality score out of the Call Generation Model SV caller representing a likelihood of a structural variant being called, x) a ref/alt insert size log likelihood ratio (medium importance for deletions, medium importance for insertions) which indicates a likelihood ratio of ref to alt based on implied insert sizes of reads, xi) a median read depth (medium importance for deletions, low importance for insertions) which indicates a median read depth over the span of a structural variant with at least a threshold MAPQ (e.g., MAPQ>20), xii) an alt forward support fraction (medium importance for deletions, low importance for insertions) which indicates a percent of nucleotide reads with perfect alignment to an alternate contiguous sequence and that have a forward orientation, xiii) an extended MAPQ standard deviation (low importance for deletions, medium importance for insertions) which indicates, on an extended MAPQ scale (e.g., max MAPQ=250), a standard deviation of MAPQ across reads with perfect alignment to an alternate contiguous sequence, xiv) a left/right median depth (low importance for deletions, low importance for insertions) which indicates median read depth in left and right flanks, respectively, and xv) split read counts (medium importance for deletions, medium importance for insertions) which indicates split read counts supporting a reference sequence and split reads counts supporting an alternate sequence. Some of these features are described in further detail above.

For a more complete list of reference-based sequencing metrics, including indications of their respective importance measures relative to different structural variants, the call refinement system 106 determines one or more of the following reference-based sequencing metrics: i) a tandem repeat length (high importance for deletions, high importance for insertions) which indicates a length of a tandem repeat sequence in a local reference spanning coordinates of an initial structural variant call (if the reference is not a tandem repeat, this metric is 0), ii) a tandem repeat ratio (high importance for deletions, high importance for insertions) which indicates a ratio or comparison between a tandem repeat length and a structural variant length in an initial structural variant call (e.g., TR length/SV length), iii) a tandem repeat match percentage (medium importance for deletions, low importance for insertions) which indicates an exactness of a match between tandem repeats in a reference sequence, iv) an alt/ref alignment score (high importance for deletions, high importance for insertions) which indicates a normalized alignment score of an alternate contiguous sequence to a reference modified with only a variant—e.g., a measure of alt contig divergence from a reference in flanking regions, v) an alt/ref alignment: SV length estimate (medium importance for deletions, high importance for insertions) which indicates an estimated total length of a deletion or an insertion based on a CIGAR string from alignment of an alternate contiguous sequence to a reference sequence modified with just variant without any soft clipping, vi) a quad reference permutation entropy (high importance for deletions, high importance for insertions) which indicates an entropy measure of quad nucleotide sequences in local a reference sequence, vii) a reference palindrome match (medium importance for deletions, medium importance for insertions) which indicates a measure of closeness to a palindrome of a local reference sequence in a structural variant region (could be predictor of chromosomal folding), viii) Levenshtein distance alt→ref (medium importance for deletions, low importance for insertions) which indicates a Levenshtein distance between an alternate contiguous sequence and a reference sequence modified with only a variant (another measure of alt contig divergence from ref in flanking regions), ix) a di palindrome permutation entropy (medium importance for deletions, low importance for insertions) which indicates an entropy measure of a di-nucleotide sequence for a palindrome (or near palindrome) section of local reference sequence, x) a tri reference permutation entropy (medium importance for deletions, high importance for insertions) which indicates an entropy measure of tri-nucleotide sequences in a local reference sequence, xi) tandem repeat permutation entropy (low importance for deletions, low importance for insertions) which indicates an entropy measure of di-nucleotide sequences in tandem repeat sections of a local reference sequence, xii) a deletion sequence alignment score (low importance for deletions, medium importance for insertions) which indicates a normalized alignment score of a deleted variant sequence relative to left/right flanks of a local reference sequence, xiii) a single reference permutation entropy (low importance for deletions, low importance for insertions) which indicates an entropy measure of single nucleotides in a local reference sequence, and xiv) a double reference permutation entropy (low importance for deletions, medium importance for insertions) which indicates an entropy measure of di-nucleotides in a local reference sequence. Some of these features are described in further detail above.

For a more complete list of variant region quality sequencing metrics, including indications of their respective importance measures relative to different structural variants, the call refinement system 106 determines one or more of the following variant region quality sequencing metrics: i) a number of soft clipped reads with high numbers of bases having low base call qualities (medium importance for deletions, medium importance for insertions) which indicates a fraction of soft clipped reads with a high number of nucleotide bases called with low base call quality (e.g., BQ<15) and ii) alternate contiguous sequences with low base call quality (low importance for deletions, low importance for insertions) which indicates, among alternate supporting reads aligned to an alternate contiguous sequence, a computation of the median base call quality (BQ) in each column and a count of median values less than a threshold (e.g., 20). These features are described in further detail above.

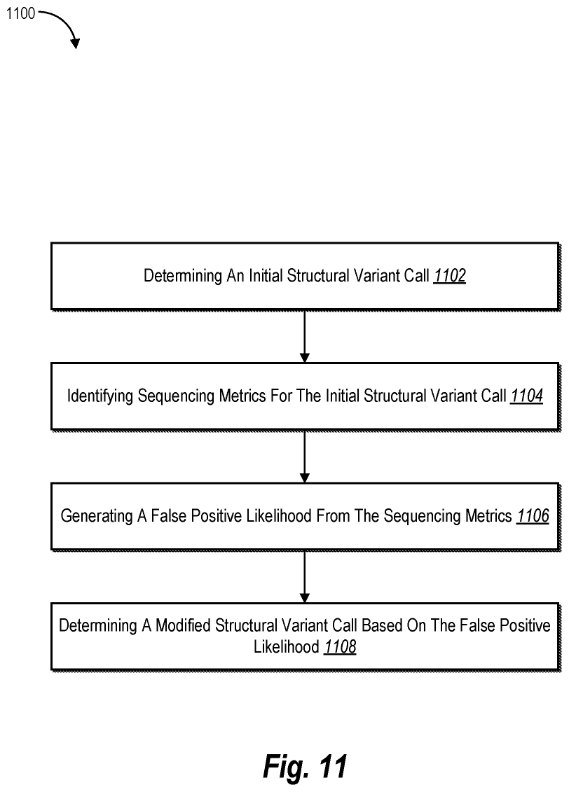

Turning now to FIG. 11, this figure illustrates an example flowchart of a series of acts of determining a modified structural variant call from a false positive likelihood using a structural variant refinement machine-learning model in accordance with one or more embodiments. While FIG. 11 illustrates acts according to one embodiment, alternative embodiments may omit, add to, reorder, and/or modify any of the acts shown in FIG. 11. The acts of FIG. 11 can be performed as part of a method. Alternatively, a non-transitory computer readable storage medium can comprise instructions that, when executed by one or more processors, cause a computing device to perform the acts depicted in FIG. 11. In still further embodiments, a system comprising at least one processor and a non-transitory computer readable medium comprising instructions that, when executed by one or more processors, cause the system to perform the acts of FIG. 11.

As shown in FIG. 11, the series of acts 1100 includes an act 1102 of determining an initial structural variant call. In particular, the act 1102 can involve determining, for one or more genomic coordinates of a genomic sample, an initial structural variant call based on nucleotide reads corresponding to the genomic sample. For example, the act 1102 can involve determining a deletion of more than a threshold number of base pairs, an insertion of more than the threshold number of base pairs, a duplication of more than the threshold number of base pairs, an inversion, a translocation, or a copy number variation (CNV). In some cases, the act 1102 involves determining a structural variant call of a number of base pairs within a threshold range of base pairs.

In addition, the series of acts 1100 includes an act 1104 of identifying sequencing metrics for the initial structural variant call. In particular, the act 1104 can involve identifying sequencing metrics corresponding to one or more of the initial structural variant call or the one or more genomic coordinates. For example, the act 1104 can involve identifying one or more of read-based sequencing metrics, reference-based sequencing metrics, or variant region quality sequencing metrics. In some cases, the act 1104 involves utilizing a call generation model to determine base calls corresponding to the one or more genomic coordinates of the genomic sample indicate a structural variant in relation to a reference genome.

Identifying read-based sequencing metrics can involve determining, for the initial structural variant call, one or more of: a base-call quality score, a fraction of nucleotide reads supporting an alternate contiguous sequence from a reference genome, a number of split nucleotide reads from the nucleotide reads corresponding to the initial structural variant call, a coverage depth of the nucleotide reads corresponding to the initial structural variant call, an additional structural variant call located within a threshold number of base pairs from the initial structural variant call within the genomic sample, an alignment of a contiguous sequence corresponding to the nucleotide reads with a reference sequence of a reference genome modified to include a structural variant corresponding to the initial structural variant call, a deletion length in nucleotide bases based on one or more soft clipped nucleotide reads, a number of the nucleotide reads exhibit a mapping quality metric that fails to satisfy a threshold mapping quality metric, insert sizes corresponding to the one or more genomic coordinates of the genomic sample, or a likelihood ratio between a reference call and an alternate call based on an insert size.

As part of the act 1104, identifying variant region quality sequencing metrics can involve determining one or more of: a number of nucleotide reads that comprise at least a threshold number of base calls and correspond to a target genomic region for the initial structural variant call or a number of nucleotide bases in an alternate contiguous sequence corresponding to the target genomic region from a reference genome for which based calls for the nucleotide reads fail to satisfy a threshold base call quality score. As a further part of the act 1104, identifying reference-based sequencing metrics can involve identifying, within one or more genomic regions of a reference genome corresponding to the one or more genomic coordinates of the genomic sample, one or more of: a tandem repeat length in nucleotide bases, or a permutation entropy of nucleotide bases, a cytosine quadruplex (C-quadruplex), a guanine quadruplex (G-quadruplex).

Further, the series of acts 1100 includes an act 1106 of generating a false positive likelihood from the sequencing metrics. In particular, the act 1106 can involve generating, utilizing a structural variant refinement machine-learning model based on the sequencing metrics, a false positive likelihood indicating a likelihood that the initial structural variant call is a false positive. For example, the act 1106 can involve determining the initial structural variant call is a false positive call or a true positive call based on the sequencing metrics. As a further example, the act 1106 can involve generating the false positive likelihood utilizing the structural variant refinement machine-learning model based on the sequencing metrics and the initial structural variant call as inputs.

Additionally, the series of acts 1100 includes an act 1108 of determining a modified structural variant call based on the false positive likelihood. In particular, the act 1108 can involve determining a modified structural variant call for the one or more genomic coordinates of the genomic sample based on the false positive likelihood. For example, the act 1108 can involve changing the initial structural variant call from a positive structural variant call to a negative structural variant call based on the initial structural variant call being the false positive call or changing the initial structural variant call from a negative structural variant call to a positive structural variant call based on the initial structural variant call being the true positive call. In some cases, the act 1108 involves correcting the initial structural variant call for the one or more genomic coordinates based on the false positive likelihood generated by the structural variant refinement machine-learning model.

In some embodiments, the series of acts 1100 includes an act of determining, from a truth dataset, a ground truth structural variant call corresponding to the modified structural variant call is incorrectly labeled as a false positive instead of a true positive based on one or more truth set nucleotide reads for the ground truth structural variant call satisfying structural variant criteria. The series of acts 1100 can also include an act of changing a label for the ground truth structural variant call from false positive to true positive. Further, the series of acts 1100 can include an act of adjusting parameters of the structural variant refinement machine-learning model based on a comparison of the modified structural variant call and the ground truth structural variant call.

In one or more embodiments, determining that a ground truth structural variant call is incorrectly labeled based on the structural variant criteria can involve: parsing a Concise Idiosyncratic Gapped Alignment Report (CIGAR) string to identify a truth set nucleotide read of the truth dataset that satisfies a threshold mapping quality metric, determining a portion of the CIGAR string comprising a starting index of a corresponding structural variant call generated by a call generation model, and determining that the starting index corresponds to a structural variant and matches a length of the corresponding structural variant call generated by the call generation model.

The methods described herein can be used in conjunction with a variety of nucleic acid sequencing techniques. Particularly applicable techniques are those wherein nucleic acids are attached at fixed locations in an array such that their relative positions do not change and wherein the array is repeatedly imaged. Embodiments in which images are obtained in different color channels, for example, coinciding with different labels used to distinguish one nucleotide base type from another are particularly applicable. In some embodiments, the process to determine the nucleotide sequence of a target nucleic acid (i.e., a nucleic acid polymer) can be an automated process. Preferred embodiments include sequencing-by-synthesis (SBS) techniques.

SBS techniques generally involve the enzymatic extension of a nascent nucleic acid strand through the iterative addition of nucleotides against a template strand. In traditional methods of SBS, a single nucleotide monomer may be provided to a target nucleotide in the presence of a polymerase in each delivery. However, in the methods described herein, more than one type of nucleotide monomer can be provided to a target nucleic acid in the presence of a polymerase in a delivery.

SBS can utilize nucleotide monomers that have a terminator moiety or those that lack any terminator moieties. Methods utilizing nucleotide monomers lacking terminators include, for example, pyrosequencing and sequencing using γ-phosphate-labeled nucleotides, as set forth in further detail below. In methods using nucleotide monomers lacking terminators, the number of nucleotides added in each cycle is generally variable and dependent upon the template sequence and the mode of nucleotide delivery. For SBS techniques that utilize nucleotide monomers having a terminator moiety, the terminator can be effectively irreversible under the sequencing conditions used as is the case for traditional Sanger sequencing which utilizes dideoxynucleotides, or the terminator can be reversible as is the case for sequencing methods developed by Solexa (now Illumina, Inc.).

SBS techniques can utilize nucleotide monomers that have a label moiety or those that lack a label moiety. Accordingly, incorporation events can be detected based on a characteristic of the label, such as fluorescence of the label; a characteristic of the nucleotide monomer such as molecular weight or charge; a byproduct of incorporation of the nucleotide, such as release of pyrophosphate; or the like. In embodiments, where two or more different nucleotides are present in a sequencing reagent, the different nucleotides can be distinguishable from each other, or alternatively, the two or more different labels can be the indistinguishable under the detection techniques being used. For example, the different nucleotides present in a sequencing reagent can have different labels and they can be distinguished using appropriate optics as exemplified by the sequencing methods developed by Solexa (now Illumina, Inc.).

Preferred embodiments include pyrosequencing techniques. Pyrosequencing detects the release of inorganic pyrophosphate (PPi) as particular nucleotides are incorporated into the nascent strand (Ronaghi, M., Karamohamed, S., Pettersson, B., Uhlen, M. and Nyren, P. (1996) “Real-time DNA sequencing using detection of pyrophosphate release.” Analytical Biochemistry 242(1), 84-9; Ronaghi, M. (2001) “Pyrosequencing sheds light on DNA sequencing.” Genome Res. 11(1), 3-11; Ronaghi, M., Uhlen, M. and Nyren, P. (1998) “A sequencing method based on real-time pyrophosphate.” Science 281(5375), 363; U.S. Pat. Nos. 6,210,891; 6,258,568 and 6,274,320, the disclosures of which are incorporated herein by reference in their entireties). In pyrosequencing, released PPi can be detected by being immediately converted to adenosine triphosphate (ATP) by ATP sulfurylase, and the level of ATP generated is detected via luciferase-produced photons. The nucleic acids to be sequenced can be attached to features in an array and the array can be imaged to capture the chemiluminescent signals that are produced due to incorporation of a nucleotides at the features of the array. An image can be obtained after the array is treated with a particular nucleotide type (e.g., A, T, C or G). Images obtained after addition of each nucleotide type will differ with regard to which features in the array are detected. These differences in the image reflect the different sequence content of the features on the array. However, the relative locations of each feature will remain unchanged in the images. The images can be stored, processed and analyzed using the methods set forth herein. For example, images obtained after treatment of the array with each different nucleotide type can be handled in the same way as exemplified herein for images obtained from different detection channels for reversible terminator-based sequencing methods.

In another exemplary type of SBS, cycle sequencing is accomplished by stepwise addition of reversible terminator nucleotides containing, for example, a cleavable or photobleachable dye label as described, for example, in WO 04/018497 and U.S. Pat. No. 7,057,026, the disclosures of which are incorporated herein by reference. This approach is being commercialized by Solexa (now Illumina Inc.), and is also described in WO 91/06678 and WO 07/123,744, each of which is incorporated herein by reference. The availability of fluorescently-labeled terminators in which both the termination can be reversed and the fluorescent label cleaved facilitates efficient cyclic reversible termination (CRT) sequencing. Polymerases can also be co-engineered to efficiently incorporate and extend from these modified nucleotides.

Preferably in reversible terminator-based sequencing embodiments, the labels do not substantially inhibit extension under SBS reaction conditions. However, the detection labels can be removable, for example, by cleavage or degradation. Images can be captured following incorporation of labels into arrayed nucleic acid features. In particular embodiments, each cycle involves simultaneous delivery of four different nucleotide types to the array and each nucleotide type has a spectrally distinct label. Four images can then be obtained, each using a detection channel that is selective for one of the four different labels. Alternatively, different nucleotide types can be added sequentially and an image of the array can be obtained between each addition step. In such embodiments, each image will show nucleic acid features that have incorporated nucleotides of a particular type. Different features are present or absent in the different images due the different sequence content of each feature. However, the relative position of the features will remain unchanged in the images. Images obtained from such reversible terminator-SBS methods can be stored, processed and analyzed as set forth herein. Following the image capture step, labels can be removed and reversible terminator moieties can be removed for subsequent cycles of nucleotide addition and detection. Removal of the labels after they have been detected in a particular cycle and prior to a subsequent cycle can provide the advantage of reducing background signal and crosstalk between cycles. Examples of useful labels and removal methods are set forth below.

In particular embodiments some or all of the nucleotide monomers can include reversible terminators. In such embodiments, reversible terminators/cleavable fluors can include fluor linked to the ribose moiety via a 3′ ester linkage (Metzker, Genome Res. 15:1767-1776 (2005), which is incorporated herein by reference). Other approaches have separated the terminator chemistry from the cleavage of the fluorescence label (Ruparel et al., Proc Natl Acad Sci USA 102: 5932-7 (2005), which is incorporated herein by reference in its entirety). Ruparel et al described the development of reversible terminators that used a small 3′ allyl group to block extension, but could easily be deblocked by a short treatment with a palladium catalyst. The fluorophore was attached to the base via a photocleavable linker that could easily be cleaved by a 30 second exposure to long wavelength UV light. Thus, either disulfide reduction or photocleavage can be used as a cleavable linker. Another approach to reversible termination is the use of natural termination that ensues after placement of a bulky dye on a dNTP. The presence of a charged bulky dye on the dNTP can act as an effective terminator through steric and/or electrostatic hindrance. The presence of one incorporation event prevents further incorporations unless the dye is removed. Cleavage of the dye removes the fluor and effectively reverses the termination. Examples of modified nucleotides are also described in U.S. Pat. Nos. 7,427,673, and 7,057,026, the disclosures of which are incorporated herein by reference in their entireties.

Additional exemplary SBS systems and methods which can be utilized with the methods and systems described herein are described in U.S. Patent Application Publication No. 2007/0166705, U.S. Patent Application Publication No. 2006/0188901, U.S. Pat. No. 7,057,026, U.S. Patent Application Publication No. 2006/0240439, U.S. Patent Application Publication No. 2006/0281109, PCT Publication No. WO 05/065814, U.S. Patent Application Publication No. 2005/0100900, PCT Publication No. WO 06/064199, PCT Publication No. WO 07/010,251, U.S. Patent Application Publication No. 2012/0270305 and U.S. Patent Application Publication No. 2013/0260372, the disclosures of which are incorporated herein by reference in their entireties.

Some embodiments can utilize detection of four different nucleotides using fewer than four different labels. For example, SBS can be performed utilizing methods and systems described in the incorporated materials of U.S. Patent Application Publication No. 2013/0079232. As a first example, a pair of nucleotide types can be detected at the same wavelength, but distinguished based on a difference in intensity for one member of the pair compared to the other, or based on a change to one member of the pair (e.g. via chemical modification, photochemical modification or physical modification) that causes apparent signal to appear or disappear compared to the signal detected for the other member of the pair. As a second example, three of four different nucleotide types can be detected under particular conditions while a fourth nucleotide type lacks a label that is detectable under those conditions, or is minimally detected under those conditions (e.g., minimal detection due to background fluorescence, etc.). Incorporation of the first three nucleotide types into a nucleic acid can be determined based on presence of their respective signals and incorporation of the fourth nucleotide type into the nucleic acid can be determined based on absence or minimal detection of any signal. As a third example, one nucleotide type can include label(s) that are detected in two different channels, whereas other nucleotide types are detected in no more than one of the channels. The aforementioned three exemplary configurations are not considered mutually exclusive and can be used in various combinations. An exemplary embodiment that combines all three examples, is a fluorescent-based SBS method that uses a first nucleotide type that is detected in a first channel (e.g. dATP having a label that is detected in the first channel when excited by a first excitation wavelength), a second nucleotide type that is detected in a second channel (e.g. dCTP having a label that is detected in the second channel when excited by a second excitation wavelength), a third nucleotide type that is detected in both the first and the second channel (e.g. dTTP having at least one label that is detected in both channels when excited by the first and/or second excitation wavelength) and a fourth nucleotide type that lacks a label that is not, or minimally, detected in either channel (e.g. dGTP having no label).

Further, as described in the incorporated materials of U.S. Patent Application Publication No. 2013/0079232, sequencing data can be obtained using a single channel. In such so-called one-dye sequencing approaches, the first nucleotide type is labeled but the label is removed after the first image is generated, and the second nucleotide type is labeled only after a first image is generated. The third nucleotide type retains its label in both the first and second images, and the fourth nucleotide type remains unlabeled in both images.

Some embodiments can utilize sequencing by ligation techniques. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. As with other SBS methods, images can be obtained following treatment of an array of nucleic acid features with the labeled sequencing reagents. Each image will show nucleic acid features that have incorporated labels of a particular type. Different features are present or absent in the different images due the different sequence content of each feature, but the relative position of the features will remain unchanged in the images. Images obtained from ligation-based sequencing methods can be stored, processed and analyzed as set forth herein. Exemplary SBS systems and methods which can be utilized with the methods and systems described herein are described in U.S. Pat. Nos. 6,969,488, 6,172,218, and 6,306,597, the disclosures of which are incorporated herein by reference in their entireties.

Some embodiments can utilize nanopore sequencing (Deamer, D. W. & Akeson, M. “Nanopores and nucleic acids: prospects for ultrarapid sequencing.” Trends Biotechnol. 18, 147-151 (2000); Deamer, D. and D. Branton, “Characterization of nucleic acids by nanopore analysis”. Acc. Chem. Res. 35:817-825 (2002); Li, J., M. Gershow, D. Stein, E. Brandin, and J. A. Golovchenko, “DNA molecules and configurations in a solid-state nanopore microscope” Nat. Mater. 2:611-615 (2003), the disclosures of which are incorporated herein by reference in their entireties). In such embodiments, the target nucleic acid passes through a nanopore. The nanopore can be a synthetic pore or biological membrane protein, such as α-hemolysin. As the target nucleic acid passes through the nanopore, each base-pair can be identified by measuring fluctuations in the electrical conductance of the pore. (U.S. Pat. No. 7,001,792; Soni, G. V. & Meller, “A. Progress toward ultrafast DNA sequencing using solid-state nanopores.” Clin. Chem. 53, 1996-2001 (2007); Healy, K. “Nanopore-based single-molecule DNA analysis.” Nanomed. 2, 459-481 (2007); Cockroft, S. L., Chu, J., Amorin, M. & Ghadiri, M. R. “A single-molecule nanopore device detects DNA polymerase activity with single-nucleotide resolution.” J. Am. Chem. Soc. 130, 818-820 (2008), the disclosures of which are incorporated herein by reference in their entireties). Data obtained from nanopore sequencing can be stored, processed and analyzed as set forth herein. In particular, the data can be treated as an image in accordance with the exemplary treatment of optical images and other images that is set forth herein.

Some embodiments can utilize methods involving the real-time monitoring of DNA polymerase activity. Nucleotide incorporations can be detected through fluorescence resonance energy transfer (FRET) interactions between a fluorophore-bearing polymerase and γ-phosphate-labeled nucleotides as described, for example, in U.S. Pat. Nos. 7,329,492 and 7,211,414 (each of which is incorporated herein by reference) or nucleotide incorporations can be detected with zero-mode waveguides as described, for example, in U.S. Pat. No. 7,315,019 (which is incorporated herein by reference) and using fluorescent nucleotide analogs and engineered polymerases as described, for example, in U.S. Pat. No. 7,405,281 and U.S. Patent Application Publication No. 2008/0108082 (each of which is incorporated herein by reference). The illumination can be restricted to a zeptoliter-scale volume around a surface-tethered polymerase such that incorporation of fluorescently labeled nucleotides can be observed with low background (Levene, M. J. et al. “Zero-mode waveguides for single-molecule analysis at high concentrations.” Science 299, 682-686 (2003); Lundquist, P. M. et al. “Parallel confocal detection of single molecules in real time.” Opt. Lett. 33, 1026-1028 (2008); Korlach, J. et al. “Selective aluminum passivation for targeted immobilization of single DNA polymerase molecules in zero-mode waveguide nano structures.” Proc. Natl. Acad. Sci. USA 105, 1176-1181 (2008), the disclosures of which are incorporated herein by reference in their entireties). Images obtained from such methods can be stored, processed and analyzed as set forth herein.

Some SBS embodiments include detection of a proton released upon incorporation of a nucleotide into an extension product. For example, sequencing based on detection of released protons can use an electrical detector and associated techniques that are commercially available from Ion Torrent (Guilford, CT, a Life Technologies subsidiary) or sequencing methods and systems described in US 2009/0026082 A1; US 2009/0127589 A1; US 2010/0137143 A1; or US 2010/0282617 A1, each of which is incorporated herein by reference. Methods set forth herein for amplifying target nucleic acids using kinetic exclusion can be readily applied to substrates used for detecting protons. More specifically, methods set forth herein can be used to produce clonal populations of amplicons that are used to detect protons.

The above SBS methods can be advantageously carried out in multiplex formats such that multiple different target nucleic acids are manipulated simultaneously. In particular embodiments, different target nucleic acids can be treated in a common reaction vessel or on a surface of a particular substrate. This allows convenient delivery of sequencing reagents, removal of unreacted reagents and detection of incorporation events in a multiplex manner. In embodiments using surface-bound target nucleic acids, the target nucleic acids can be in an array format. In an array format, the target nucleic acids can be typically bound to a surface in a spatially distinguishable manner. The target nucleic acids can be bound by direct covalent attachment, attachment to a bead or other particle or binding to a polymerase or other molecule that is attached to the surface. The array can include a single copy of a target nucleic acid at each site (also referred to as a feature) or multiple copies having the same sequence can be present at each site or feature. Multiple copies can be produced by amplification methods such as, bridge amplification or emulsion PCR as described in further detail below.

The methods set forth herein can use arrays having features at any of a variety of densities including, for example, at least about 10 features/cm2, 100 features/cm2, 500 features/cm2, 1,000 features/cm2, 5,000 features/cm2, 10,000 features/cm2, 50,000 features/cm2, 100,000 features/cm2, 1,000,000 features/cm2, 5,000,000 features/cm2, or higher.

An advantage of the methods set forth herein is that they provide for rapid and efficient detection of a plurality of target nucleic acid in parallel. Accordingly the present disclosure provides integrated systems capable of preparing and detecting nucleic acids using techniques known in the art such as those exemplified above. Thus, an integrated system of the present disclosure can include fluidic components capable of delivering amplification reagents and/or sequencing reagents to one or more immobilized DNA fragments, the system comprising components such as pumps, valves, reservoirs, fluidic lines and the like. A flow cell can be configured and/or used in an integrated system for detection of target nucleic acids. Exemplary flow cells are described, for example, in US 2010/0111768 A1 and U.S. Ser. No. 13/273,666, each of which is incorporated herein by reference. As exemplified for flow cells, one or more of the fluidic components of an integrated system can be used for an amplification method and for a detection method. Taking a nucleic acid sequencing embodiment as an example, one or more of the fluidic components of an integrated system can be used for an amplification method set forth herein and for the delivery of sequencing reagents in a sequencing method such as those exemplified above. Alternatively, an integrated system can include separate fluidic systems to carry out amplification methods and to carry out detection methods. Examples of integrated sequencing systems that are capable of creating amplified nucleic acids and also determining the sequence of the nucleic acids include, without limitation, the MiSeq™ platform (Illumina, Inc., San Diego, CA) and devices described in U.S. Ser. No. 13/273,666, which is incorporated herein by reference.

The sequencing system described above sequences nucleic acid polymers present in samples received by a sequencing device. As defined herein, “sample” and its derivatives, is used in its broadest sense and includes any specimen, culture and the like that is suspected of including a target. In some embodiments, the sample comprises DNA, RNA, PNA, LNA, chimeric or hybrid forms of nucleic acids. The sample can include any biological, clinical, surgical, agricultural, atmospheric or aquatic-based specimen containing one or more nucleic acids. The term also includes any isolated nucleic acid sample such a genomic DNA, fresh-frozen or formalin-fixed paraffin-embedded nucleic acid specimen. It is also envisioned that the sample can be from a single individual, a collection of nucleic acid samples from genetically related members, nucleic acid samples from genetically unrelated members, nucleic acid samples (matched) from a single individual such as a tumor sample and normal tissue sample, or sample from a single source that contains two distinct forms of genetic material such as maternal and fetal DNA obtained from a maternal subject, or the presence of contaminating bacterial DNA in a sample that contains plant or animal DNA. In some embodiments, the source of nucleic acid material can include nucleic acids obtained from a newborn, for example as typically used for newborn screening.

The nucleic acid sample can include high molecular weight material such as genomic DNA (gDNA). The sample can include low molecular weight material such as nucleic acid molecules obtained from FFPE or archived DNA samples. In another embodiment, low molecular weight material includes enzymatically or mechanically fragmented DNA. The sample can include cell-free circulating DNA. In some embodiments, the sample can include nucleic acid molecules obtained from biopsies, tumors, scrapings, swabs, blood, mucus, urine, plasma, semen, hair, laser capture micro-dissections, surgical resections, and other clinical or laboratory obtained samples. In some embodiments, the sample can be an epidemiological, agricultural, forensic or pathogenic sample. In some embodiments, the sample can include nucleic acid molecules obtained from an animal such as a human or mammalian source. In another embodiment, the sample can include nucleic acid molecules obtained from a non-mammalian source such as a plant, bacteria, virus or fungus. In some embodiments, the source of the nucleic acid molecules may be an archived or extinct sample or species.

Further, the methods and compositions disclosed herein may be useful to amplify a nucleic acid sample having low-quality nucleic acid molecules, such as degraded and/or fragmented genomic DNA from a forensic sample. In one embodiment, forensic samples can include nucleic acids obtained from a crime scene, nucleic acids obtained from a missing persons DNA database, nucleic acids obtained from a laboratory associated with a forensic investigation or include forensic samples obtained by law enforcement agencies, one or more military services or any such personnel. The nucleic acid sample may be a purified sample or a crude DNA containing lysate, for example derived from a buccal swab, paper, fabric or other substrate that may be impregnated with saliva, blood, or other bodily fluids. As such, in some embodiments, the nucleic acid sample may comprise low amounts of, or fragmented portions of DNA, such as genomic DNA. In some embodiments, target sequences can be present in one or more bodily fluids including but not limited to, blood, sputum, plasma, semen, urine and serum. In some embodiments, target sequences can be obtained from hair, skin, tissue samples, autopsy or remains of a victim. In some embodiments, nucleic acids including one or more target sequences can be obtained from a deceased animal or human. In some embodiments, target sequences can include nucleic acids obtained from non-human DNA such a microbial, plant or entomological DNA. In some embodiments, target sequences or amplified target sequences are directed to purposes of human identification. In some embodiments, the disclosure relates generally to methods for identifying characteristics of a forensic sample. In some embodiments, the disclosure relates generally to human identification methods using one or more target specific primers disclosed herein or one or more target specific primers designed using the primer design criteria outlined herein. In one embodiment, a forensic or human identification sample containing at least one target sequence can be amplified using any one or more of the target-specific primers disclosed herein or using the primer criteria outlined herein.

The components of the call refinement system 106 can include software, hardware, or both. For example, the components of the call refinement system 106 can include one or more instructions stored on a computer-readable storage medium and executable by processors of one or more computing devices (e.g., the client device 108). When executed by the one or more processors, the computer-executable instructions of the call refinement system 106 can cause the computing devices to perform the bubble detection methods described herein. Alternatively, the components of the call refinement system 106 can comprise hardware, such as special purpose processing devices to perform a certain function or group of functions. Additionally, or alternatively, the components of the call refinement system 106 can include a combination of computer-executable instructions and hardware.

Furthermore, the components of the call refinement system 106 performing the functions described herein with respect to the call refinement system 106 may, for example, be implemented as part of a stand-alone application, as a module of an application, as a plug-in for applications, as a library function or functions that may be called by other applications, and/or as a cloud-computing model. Thus, components of the call refinement system 106 may be implemented as part of a stand-alone application on a personal computing device or a mobile device. Additionally, or alternatively, the components of the call refinement system 106 may be implemented in any application that provides sequencing services including, but not limited to Illumina BaseSpace, Illumina DRAGEN, Illumina DRAGEN SV Caller, or Illumina TruSight software. “Illumina,” “BaseSpace,” “DRAGEN,” “DRAGEN SV,” “DRAGEN SV Caller,” and “TruSight,” are either registered trademarks or trademarks of Illumina, Inc. in the United States and/or other countries.

Embodiments of the present disclosure may comprise or utilize a special purpose or general-purpose computer including computer hardware, such as, for example, one or more processors and system memory, as discussed in greater detail below. Embodiments within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., a memory, etc.), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (SSDs) (e.g., based on RAM), Flash memory, phase-change memory (PCM), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a NIC), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed at a processor, cause a general purpose computer, special purpose computer, or special purpose processing device to perform a certain function or group of functions. In some embodiments, computer-executable instructions are executed on a general-purpose computer to turn the general-purpose computer into a special purpose computer implementing elements of the disclosure. The computer executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Embodiments of the present disclosure can also be implemented in cloud computing environments. In this description, “cloud computing” is defined as a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (SaaS), Platform as a Service (PaaS), and Infrastructure as a Service (IaaS). A cloud-computing model can also be deployed using different deployment models such as private cloud, community cloud, public cloud, hybrid cloud, and so forth. In this description and in the claims, a “cloud-computing environment” is an environment in which cloud computing is employed.

FIG. 12 illustrates a block diagram of a computing device 1200 that may be configured to perform one or more of the processes described above. One will appreciate that one or more computing devices such as the computing device 1200 may implement the call refinement system 106 and the sequencing system 104. As shown by FIG. 12, the computing device 1200 can comprise a processor 1202, a memory 1204, a storage device 1206, an I/O interface 1208, and a communication interface 1210, which may be communicatively coupled by way of a communication infrastructure 1212. In certain embodiments, the computing device 1200 can include fewer or more components than those shown in FIG. 12. The following paragraphs describe components of the computing device 1200 shown in FIG. 12 in additional detail.

In one or more embodiments, the processor 1202 includes hardware for executing instructions, such as those making up a computer program. As an example, and not by way of limitation, to execute instructions for dynamically modifying workflows, the processor 1202 may retrieve (or fetch) the instructions from an internal register, an internal cache, the memory 1204, or the storage device 1206 and decode and execute them. The memory 1204 may be a volatile or non-volatile memory used for storing data, metadata, and programs for execution by the processor(s). The storage device 1206 includes storage, such as a hard disk, flash disk drive, or other digital storage device, for storing data or instructions for performing the methods described herein.

The I/O interface 1208 allows a user to provide input to, receive output from, and otherwise transfer data to and receive data from computing device 1200. The I/O interface 1208 may include a mouse, a keypad or a keyboard, a touch screen, a camera, an optical scanner, network interface, modem, other known I/O devices or a combination of such I/O interfaces. The I/O interface 1208 may include one or more devices for presenting output to a user, including, but not limited to, a graphics engine, a display (e.g., a display screen), one or more output drivers (e.g., display drivers), one or more audio speakers, and one or more audio drivers. In certain embodiments, the I/O interface 1208 is configured to provide graphical data to a display for presentation to a user. The graphical data may be representative of one or more graphical user interfaces and/or any other graphical content as may serve a particular implementation.

The communication interface 1210 can include hardware, software, or both. In any event, the communication interface 1210 can provide one or more interfaces for communication (such as, for example, packet-based communication) between the computing device 1200 and one or more other computing devices or networks. As an example, and not by way of limitation, the communication interface 1210 may include a network interface controller (NIC) or network adapter for communicating with an Ethernet or other wire-based network or a wireless NIC (WNIC) or wireless adapter for communicating with a wireless network, such as a WI-FI.

Additionally, the communication interface 1210 may facilitate communications with various types of wired or wireless networks. The communication interface 1210 may also facilitate communications using various communication protocols. The communication infrastructure 1212 may also include hardware, software, or both that couples components of the computing device 1200 to each other. For example, the communication interface 1210 may use one or more networks and/or protocols to enable a plurality of computing devices connected by a particular infrastructure to communicate with each other to perform one or more aspects of the processes described herein. To illustrate, the sequencing process can allow a plurality of devices (e.g., a client device, sequencing device, and server device(s)) to exchange information such as sequencing data and error notifications.

In the foregoing specification, the present disclosure has been described with reference to specific exemplary embodiments thereof. Various embodiments and aspects of the present disclosure(s) are described with reference to details discussed herein, and the accompanying drawings illustrate the various embodiments. The description above and drawings are illustrative of the disclosure and are not to be construed as limiting the disclosure. Numerous specific details are described to provide a thorough understanding of various embodiments of the present disclosure.

The present disclosure may be embodied in other specific forms without departing from its spirit or essential characteristics. The described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel with one another or in parallel with different instances of the same or similar steps/acts. The scope of the present application is, therefore, indicated by the appended claims rather than by the foregoing description. All changes that come within the meaning and range of equivalency of the claims are to be embraced within their scope.

We claim:

1. A system comprising:
at least one processor; and
a non-transitory computer readable medium comprising instructions that, when executed by the at least one processor, cause the system to:
determine, for one or more genomic coordinates of a genomic sample, an initial structural variant call based on nucleotide reads corresponding to the genomic sample;
identify sequencing metrics corresponding to one or more of the initial structural variant call or the one or more genomic coordinates;
generate, utilizing a structural variant refinement machine-learning model based on the sequencing metrics, a false positive likelihood indicating a likelihood that the initial structural variant call is a false positive; and
determine a modified structural variant call for the one or more genomic coordinates of the genomic sample based on the false positive likelihood.

2. The system of claim 1, further comprising instructions that, when executed by the at least one processor, cause the system to determine the initial structural variant call by determining a deletion of more than a threshold number of base pairs, an insertion of more than the threshold number of base pairs, a duplication of more than the threshold number of base pairs, an inversion, a translocation, or a copy number variation (CNV).

3. The system of claim 1, further comprising instructions that, when executed by the at least one processor, cause the system to determine the initial structural variant call by determining a structural variant call of a number of base pairs within a threshold range of base pairs.

4. The system of claim 1, further comprising instructions that, when executed by the at least one processor, cause the system to identify the sequencing metrics corresponding to the initial structural variant call by identifying one or more of read-based sequencing metrics, reference-based sequencing metrics, or variant region quality sequencing metrics.

5. The system of claim 4, further comprising instructions that, when executed by the at least one processor, cause the system to identify the read-based sequencing metrics by determining, for the initial structural variant call, one or more of:
one or more base-call quality scores;
a fraction of nucleotide reads supporting an alternate contiguous sequence from a reference genome;
a number of split nucleotide reads from the nucleotide reads corresponding to the initial structural variant call;
a coverage depth of the nucleotide reads corresponding to the initial structural variant call;
an additional structural variant call located within a threshold number of base pairs from the initial structural variant call within the genomic sample;
an alignment of a contiguous sequence corresponding to the nucleotide reads with a reference sequence of a reference genome modified to include a structural variant corresponding to the initial structural variant call;
a deletion length in nucleotide bases based on one or more soft clipped nucleotide reads;
a number of the nucleotide reads exhibiting a mapping quality metric that fails to satisfy a threshold mapping quality metric;
an insert size representing a length of nucleotide-read fragments corresponding to the initial structural variant call; or
a structural-variant likelihood representing a ratio of the initial structural variant call to a reference call for the one or more genomic coordinates based on the insert size.

6. The system of claim 4, further comprising instructions that, when executed by the at least one processor, cause the system to identify the variant region quality sequencing metrics by determining one or more of:
a number of nucleotide reads that comprise at least a threshold number of base calls and correspond to a target genomic region for the initial structural variant call; or
a number of nucleotide bases in an alternate contiguous sequence corresponding to the target genomic region from a reference genome for which base calls for the nucleotide reads fail to satisfy a threshold base call quality score.

7. The system of claim 4, further comprising instructions that, when executed by the at least one processor, cause the system to identify the reference-based sequencing metrics by identifying, within one or more genomic regions of a reference genome corresponding to the one or more genomic coordinates of the genomic sample, one or more of:
a tandem repeat length in nucleotide bases;
a permutation entropy of nucleotide bases;
a cytosine quadruplex (C-quadruplex); or
a guanine quadruplex (G-quadruplex).

8. The system of claim 1, further comprising instructions that, when executed by the at least one processor, cause the system to:
generate the false positive likelihood by determining the initial structural variant call is a false positive call or a true positive call based on the sequencing metrics; and
determine the modified structural variant call by:
changing the initial structural variant call from a positive structural variant call to a negative structural variant call based on the initial structural variant call being the false positive call; or
changing the initial structural variant call from a negative structural variant call to a positive structural variant call based on the initial structural variant call being the true positive call.

9. A computer-implemented method comprising:
determining, for one or more genomic coordinates of a genomic sample, an initial structural variant call based on nucleotide reads corresponding to the genomic sample;
identifying sequencing metrics corresponding to one or more of the initial structural variant call or the one or more genomic coordinates;
generating, utilizing a structural variant refinement machine-learning model based on the sequencing metrics, a false positive likelihood indicating a likelihood that the initial structural variant call is a false positive; and
determining a modified structural variant call for the one or more genomic coordinates of the genomic sample based on the false positive likelihood.

10. The computer-implemented method of claim 9, wherein:
determining the initial structural variant call comprises utilizing a call generation model to determine base calls corresponding to the one or more genomic coordinates of the genomic sample indicate a structural variant in relation to a reference genome; and
determining the modified structural variant call comprises correcting the initial structural variant call for the one or more genomic coordinates based on the false positive likelihood generated by the structural variant refinement machine-learning model.

11. The computer-implemented method of claim 9, wherein identifying the sequencing metrics corresponding to the initial structural variant call comprises identifying one or more of read-based sequencing metrics, reference-based sequencing metrics, or variant region quality sequencing metrics.

12. The computer-implemented method of claim 9, wherein identifying the sequencing metrics comprises determining, for the initial structural variant call, one or more of:
one or more base-call quality scores;
a fraction of nucleotide reads supporting an alternate contiguous sequence from a reference genome;
a number of split nucleotide reads from the nucleotide reads corresponding to the initial structural variant call;
a coverage depth of the nucleotide reads corresponding to the initial structural variant call;
an additional structural variant call located within a threshold number of base pairs from the initial structural variant call within the genomic sample;
an alignment of a contiguous sequence corresponding to the nucleotide reads with a reference sequence of a reference genome modified to include a structural variant corresponding to the initial structural variant call;
a deletion length in nucleotide bases based on one or more soft clipped nucleotide reads;
a number of the nucleotide reads exhibiting a mapping quality metric that fails to satisfy a threshold mapping quality metric;
an insert size representing a length of nucleotide-read fragments corresponding to the initial structural variant call; or
a structural-variant likelihood representing a ratio of the initial structural variant call to a reference call for the one or more genomic coordinates based on the insert size.

13. The computer-implemented method of claim 9, wherein identifying the sequencing metrics comprises determining one or more of:
a number of nucleotide reads that comprise at least a threshold number of base calls and correspond to a target genomic region for the initial structural variant call; or
a number of nucleotide bases in an alternate contiguous sequence corresponding to the target genomic region from a reference genome for which based calls for the nucleotide reads fail to satisfy a threshold base call quality score.

14. The computer-implemented method of claim 9, wherein identifying the sequencing metrics comprises identifying, within one or more genomic regions of a reference genome corresponding to the one or more genomic coordinates of the genomic sample, one or more of:
a tandem repeat length in nucleotide bases;
a permutation entropy of nucleotide bases;
a cytosine quadruplex (C-quadruplex); or
a guanine quadruplex (G-quadruplex).

15. A non-transitory computer readable medium comprising instructions that, when executed by at least one processor, cause a computing device to:
determine, for one or more genomic coordinates of a genomic sample, an initial structural variant call based on nucleotide reads corresponding to the genomic sample;
identify sequencing metrics corresponding to one or more of the initial structural variant call or the one or more genomic coordinates;
generate, utilizing a structural variant refinement machine-learning model based on the sequencing metrics, a false positive likelihood indicating a likelihood that the initial structural variant call is a false positive; and
determine a modified structural variant call for the one or more genomic coordinates of the genomic sample based on the false positive likelihood.

16. The non-transitory computer readable medium of claim 15, wherein the structural variant refinement machine-learning model comprises one or more gradient boosted decision trees.

17. The non-transitory computer readable medium of claim 15, further comprising instructions that, when executed by the at least one processor, cause the computing device to:
generate the false positive likelihood by determining the initial structural variant call is a false positive call or a true positive call based on the sequencing metrics; and
determine the modified structural variant call by:
changing the initial structural variant call from a positive structural variant call to a negative structural variant call based on the initial structural variant call being the false positive call; or
changing the initial structural variant call from a negative structural variant call to a positive structural variant call based on the initial structural variant call being the true positive call.

18. The non-transitory computer readable medium of claim 15, further comprising instructions that, when executed by the at least one processor, cause the computing device to:
determine, from a truth dataset, a ground truth structural variant call corresponding to the modified structural variant call is incorrectly labeled as a false positive instead of a true positive based on one or more truth set nucleotide reads for the ground truth structural variant call satisfying structural variant criteria;
change a label for the ground truth structural variant call from false positive to true positive; and
adjust parameters of the structural variant refinement machine-learning model based on a comparison of the modified structural variant call and the ground truth structural variant call.

19. The non-transitory computer readable medium of claim 18, further comprising instructions that, when executed by the at least one processor, cause the computing device to determine the ground truth structural variant call is incorrectly labeled based on the structural variant criteria by:
parsing a Concise Idiosyncratic Gapped Alignment Report (CIGAR) string to identify a truth set nucleotide read of the truth dataset that satisfies a threshold mapping quality metric;
determining a portion of the CIGAR string comprising a starting index of a corresponding structural variant call generated by a call generation model; and
determining that the starting index corresponds to a structural variant and matches a length of the corresponding structural variant call generated by the call generation model.

20. The non-transitory computer readable medium of claim 15, further comprising instructions that, when executed by the at least one processor, cause the computing device to generate the false positive likelihood utilizing the structural variant refinement machine-learning model based on the sequencing metrics and the initial structural variant call as inputs.
